SEQUENCE LISTING (14 sequences)

SEQ ID NO: 1 - DNA, 22 bp, synthetic construct
  source 1..22: mol_type = other DNA
ctggctcaggccccgccccact

SEQ ID NO: 2 - DNA, 27 bp, synthetic construct
  source 1..27: mol_type = other DNA
cttcccccacctcgctcctccctccat

SEQ ID NO: 3 - DNA, 23 bp, synthetic construct
  source 1..23: mol_type = other DNA
ctggcttccacggtccccgacac

SEQ ID NO: 4 - DNA, 28 bp, synthetic construct
  source 1..28: mol_type = other DNA
cccaagacaccctcctcatttggaaact

SEQ ID NO: 5 - DNA, 26 bp, synthetic construct
  source 1..26: mol_type = other DNA
ctcagtgctggagggtgctcgtctta

SEQ ID NO: 6 - DNA, 24 bp, synthetic construct
  source 1..24: mol_type = other DNA
aggctcggtgacatctgctcctcc

SEQ ID NO: 7 - DNA, 27 bp, synthetic construct
  source 1..27: mol_type = other DNA
ccctttgtccctatgaccagtctcagt

SEQ ID NO: 8 - DNA, 23 bp, synthetic construct
  source 1..23: mol_type = other DNA
gggatggctgcaggcctggacct

SEQ ID NO: 9 - DNA, 30 bp, synthetic construct
  source 1..30: mol_type = other DNA
tgtgagcatgtatgcatatctgtagaccca

SEQ ID NO: 10 - DNA, 22 bp, synthetic construct
  source 1..22: mol_type = other DNA
aagccagggcctcgctgtgctt

SEQ ID NO: 11 - DNA, 24 bp, synthetic construct
  source 1..24: mol_type = other DNA
tgatgacagacgcagcccagagcc

SEQ ID NO: 12 - DNA, 28 bp, synthetic construct
  source 1..28: mol_type = other DNA
gcacgtgtgggtatgaatagcaacagca

SEQ ID NO: 13 - DNA, 25 bp, synthetic construct
  source 1..25: mol_type = other DNA
tgctcactgtggttctcagcccgtt

SEQ ID NO: 14 - DNA, 26 bp, synthetic construct
  source 1..26: mol_type = other DNA
gtctgcaggtgctgtctgtctggctt

(19) United States
(12) Patent Application Publication
(10) Pub. No.: US 2026/0358350 A1
(43) Pub. Date: Nov. 12, 2026

(54) SURROGATE BIOMARKERS FOR MUCOPOLYSACCHARIDOSIS TYPE IVA

(71) Applicant: The Nemours Foundation, Jacksonville, FL (US)

(72) Inventors: Shunji TOMATSU, Wilmington, DE (US); Yasuhiko AGO, Wilmington, DE (US)

(21) Appl. No.: 19/665,490

(22) Filed: May 1, 2026

Related U.S. Application Data
(60) Provisional application No. 63/798,917, filed on May 2, 2025.

(51) Int. Cl. G01N 33/68; G01N 33/543

(52) U.S. Cl. CPC G01N 33/6893; G01N 33/543; G01N 2800/04

(57) ABSTRACT

Provided and described herein are and systems methods for using NT-proCNP in the identification of skeletal growth failure in an individual having Mucopolysaccharidoses (MPS) Type IVA.

### CROSS-REFERENCE TO RELATED APPLICATIONS

This application claims the benefit of priority to U.S. Provisional Patent Application No. 63/798,917, filed on May 2, 2025, the entire contents of which is hereby incorporated by reference in their entirety.

### STATEMENT OF GOVERNMENTAL INTEREST

The invention described herein was supported by the United States National Institutes of Health Grant No. 1-R01-HD102545-01. The United States government has certain rights in the invention.

### INCORPORATION BY REFERENCE OF SEQUENCE LISTING

The instant application contains a Sequence Listing which has been submitted electronically in XML file format and is hereby incorporated by reference in its entirety. The XML copy, created on Apr. 28, 2026, is named 045009-0072-US-00-0000040_SL.xml and is 13,426 bytes in size.

### BACKGROUND

Mucopolysaccharidoses (MPS) are a group of inherited lysosomal storage disorders caused by deficiencies in enzymes required to degrade glycosaminoglycans (GAGs), which are integral to connective tissue. The inability to break down GAGs results in accumulation in multiple tissues and organs, leading to systemic dysfunction. Depending on the specific enzyme deficiency, MPS are classified into several subtypes. In some instances, a phenomenon known as pseudodeficiency can potentially complicate diagnosis and prognosis, highlighting the importance of biomarkers that accurately reflect the stage and prognosis of the disease.

Among these, MPS IVA (Morquio A syndrome) is characterized by a deficiency in N-acetylgalactosamine-6-sulfate sulfatase (GALNS) enzyme, which leads to the ac-cumulation of keratan sulfate (KS) and chondroitin-6-sulfate (C6S). MPS IVA is a rare genetic disorder that causes growth impairment and skeletal abnormalities with an estimated incidence of 0.11 to 1.32 per 100,000 live births, depending on the region. The resultant skeletal dysplasia is primarily driven by the accumulation of GAGs (KS and C6S), which disrupts the function of chondrocytes in the growth plates, impairs cartilage matrix development, and inhibits bone matrix mineralization. The severity of bone and cartilage damage differs by the severity of the phenotype (attenuated or severe) and the type of bone affected. Bone growth by endochondral ossification is affected more severely than other types of MPS. Thus, it is a type of skeletal dysplasia affecting bone and cartilage growth. The hallmark feature of MPS IVA is progressive and characterized by incomplete or defective endochondral ossification and a successive imbalance of growth. The skeletal-related symptoms of MPS IVA include prominent forehead, abnormal face with a large mandible, disproportionate short-trunk dwarfism with short neck, cervical spine instability with odontoid hypoplasia, cervical spinal cord compression, pectus carinatum, tracheal deviation and obstruction, restrictive lung, flaring of the rib cage, kyphoscoliosis, platyspondyly, hip dysplasia with coxa valga, genu valgum, hypermobile joints, waddling gait, and pes planus. The degree of imbalance in growth between bone and other organs and tissues contributes to the unique skeletal dysplasia and clinical severity, including the narrowing of the trachea. Respiratory failure has been shown as the primary cause of death in patients (63%), followed by cardiac failure (11%), post-traumatic organ failure (11%), complications of surgery (11%), and myocardial infarction (4%).

### SUMMARY

Current clinical management of Mucopolysaccharidosis Type IVA (MPS IVA) relies heavily on measuring urinary keratan sulfate (KS) to monitor disease progression and therapeutic response. However, urinary KS primarily reflects localized glycosaminoglycan (GAG) storage and clearance in the kidneys rather than systemic skeletal burden. During enzyme replacement therapy (ERT), urinary KS levels drop significantly because the delivered enzyme can preferentially digest renal KS, resulting in an artifact and/or misleading surrogate endpoint. This reduction generally fails to correlate with clinical improvement in skeletal lesions and growth failure, leaving clinicians without a reliable surrogate biomarker to accurately assess clinical status and/or efficacy outcomes in MPS IVA.

Accordingly, provided and described herein are methods and systems for identifying and treating skeletal growth failure in Mucopolysaccharidosis Type IVA (MPS IVA) based on the foundational discovery that N-terminal pro-C-type natriuretic peptide (NT-proCNP) is elevated in MPS IVA and negatively correlated with height z-score—one of the most reliable indicators of skeletal growth failure—thereby overcoming the monitoring failures of traditional assays. To operationalize this discovery, provided herein is an automated clinical decision-support systems (CDSS) and methods configured to asynchronously extract a patient's NT-proCNP and chronological age digital payloads, executing a logic gate to trigger an interruptive clinical alert when the concentration breaches a dynamic, healthy age- and sex-matched reference concentration (e.g., ≥2.0 standard deviations). By actively detecting and identifying this state of skeletal growth failure, the disclosure further provides integrated methods of treatment comprising administering a therapeutically effective amount of a C-type natriuretic peptide (CNP) analog to an MPS IVA patient for overcoming the underlying physiological resistance.

In some embodiments, provide herein are methods of identifying skeletal growth failure in an individual having Mucopolysaccharidoses (MPS) Type IVA, the method comprising: obtaining a biological sample from the individual; detecting the presence of N-terminal pro-C-type natriuretic peptide (NT-proCNP) polypeptide and generating a measured concentration of NT-proCNP polypeptide in the biological sample; and identifying the individual as having skeletal growth failure or an increased risk of skeletal growth failure if the measured concentration of NT-proCNP is greater than an age- and sex-matched reference concentration, the age- and sex-matched reference concentration comprising the mean concentration of NT-proCNP polypeptide in age- and sex-matched healthy individuals.

In certain embodiments, the biological sample is a blood plasma sample.

In certain embodiments, the presence of N-terminal pro-C-type natriuretic peptide (NT-proCNP) polypeptide is detected by an affinity-based detection assay. In certain embodiments, the affinity-based detection assay is an ELISA.

In certain embodiments, the individual is identified as having skeletal growth failure or increased risk of skeletal growth failure if the measured concentration of NT-proCNP is equal to or greater than 1.0 standard deviations above the reference concentration. In certain embodiments, the individual is identified as having skeletal growth failure or increased risk of skeletal growth failure if the measured concentration of NT-proCNP is equal to or greater than 2.0 standard deviations above the reference concentration.

In some embodiments, provided herein are methods of treating skeletal growth failure in an individual having MPS IVA, the method comprising: measuring a concentration of N-terminal pro-C-type natriuretic peptide (NT-proCNP) in a blood plasma sample obtained from the individual; determining that the concentration of NT-proCNP is equal to or greater than 1.0 standard deviations above an age- and sex-matched reference concentration; and administering to the patient an effective amount of a C-type natriuretic peptide (CNP) analog, the CNP analogue comprising vosoritide or navepegritide, and the CNP analog being administered based on the determination that the measured NT-proCNP concentration is equal to or greater than the 1.0 standard deviation above the age- and sex-matched reference concentration.

In certain embodiments, the measured concentration of NT-proCNP is equal to o In certain embodiments, the concentration of N-terminal pro-C-type natriuretic peptide (NT-proCNP) polypeptide is measured by an affinity-based detection assay. In certain embodiments, the affinity-based detection assay is an ELISA.

In certain embodiments, the individual has received and/or is concurrently receiving elosulfase alfa. In certain embodiments, the CNP analogue reduces the concentration of NT-proCNP in the individual.

In some embodiments, provided herein are clinical decision-support (CDS) systems for identifying skeletal growth failure in Mucopolysaccharidosis Type IVA (MPS IVA), the systems comprising: (a) a network communication interface configured to obtain laboratory data packets from a clinical data repository for a patient; (b) a processor; and (c) a non-transitory computer-readable memory storing instructions that, when executed by the processor, cause the system to: (i) extract a first digital payload and a second digital payload from the clinical data repository, the first digital payload quantifying a plasma N-terminal pro-C-type natriuretic peptide (NT-proCNP) concentration for the patient, and the second digital payload identifying a chronological age of the patient; (ii) query an age-stratified reference data structure using the second digital payload to retrieve an age- and sex-matched reference concentration; (iii) execute a clinical logic gate that detects a skeletal growth failure state when the plasma NT-proCNP concentration is greater than the age- and sex-matched reference concentration; and (iv) generate a network control signal to trigger an interruptive clinical alert on a clinical endpoint device based on the detected skeletal growth failure state.

In certain embodiments, the clinical logic gate that detects a skeletal growth failure state when the plasma NT-proCNP concentration is equal to or greater than 2.0 standard deviations above the age- and sex-matched reference concentration.

In certain embodiments, the network communication interface is configured to asynchronously ingest the laboratory data packets into the clinical data repository. In certain embodiments, the network control signal modifies a physical data structure within the clinical data repository by automatically appending a classification tag to an electronic medical record of the patient, and transmitting an asynchronous network control signal to a clinical endpoint device to render the interruptive clinical alert.

In certain embodiments, the network communication interface is configured to access the clinical data repository. In certain embodiments, the network control signal is configured to modify rendering instructions of a graphical user interface (GUI) to overlay the interruptive clinical alert on an electronic medical record. In certain embodiments, the instructions further cause the system to transmit an application programming interface (API) request to persistently store a classification tag in the clinical data repository.

In certain embodiments, the clinical data repository comprises a backend database server of an electronic health record (EHR) system, a laboratory information system (LIS), or a health information exchange (HIE). In certain embodiments, the age- and sex-matched reference concentration comprises the mean concentration of NT-proCNP polypeptide in age- and sex-matched healthy individuals.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein) of which:

FIG. 1 shows the Z-score of each patient's height under 20 years of age, demonstrating a significant negative correlation with age. Each data point represents the height Z-score at the time the patient's height was first recorded during the study period. Red round dots indicate patients who received enzyme replacement therapy (ERT), gray triangular dots indicate patients who never received ERT, and purple square dots indicate patients who received hematopoietic stem cell transplantation (HSCT).

FIG. 2 shows the correlation between age, NT-proCNP levels, and height in patients with MPS IVA. The left figure displays male patients and the right figure displays female patients. Round dots indicate patients treated with enzyme replacement therapy (ERT), triangle markers indicate patients never treated with ERT, and square markers indicate patients treated with hematopoietic stem cell transplantation (HSCT). Filled markers represent patients whose height at the time of blood sample collection was recorded. The color of each marker represents the Z-score of each patient's height, calculated using the 2022 CDC growth chart. Black dotted lines indicate the median, 5th, and 95th percentiles for healthy controls. When a patient has two data points, these are connected by a green dotted line.

FIG. 3 shows the relationship between each patient's plasma NT-proCNP level, age, and subsequent height growth rate (height velocity). Height velocity was calculated from two height measurements taken 7 to 20 months apart. Each dot represents the patient's age at the time of the first height measurement (horizontal axis) and the corresponding subsequent height velocity (vertical axis). Round dots indicate patients who continuously received enzyme replacement therapy (ERT) until the second height measurement, the square dot represents a patient who received hematopoietic stem cell transplantation (HSCT) (M08), and the triangular dot indicates a patient who never received ERT until the second height measurement (M35). Larger round dots represent the attenuated phenotype and smaller round dots represent the severe phenotype. Dot fill color corresponds to the plasma NT-proCNP level at the first height measurement. The light blue line shows the 50th percentile for height velocity among male MPS IVA patients, the pink line shows the 50th percentile for female MPS IVA patients, and the gray dotted line shows the 50th percentile for height velocity among healthy males and females.

FIG. 4 shows glycosaminoglycan levels in urine and blood, including mono-sulfated keratan sulfate (KS), di-sulfated KS, the KS ratio (di-sulfated KS to total KS), and chondroitin-6-sulfate (C6S) levels, summarized in scatter plots. The horizontal axes indicate the age (years) of each patient or control. For urinary mono-sulfated and di-sulfated KS levels, the vertical axes use a base-10 logarithmic scale. Round red dots indicate patients who ever received enzyme replacement therapy (ERT), purple squares indicate patients who received hematopoietic stem cell transplantation (HSCT), gray triangles represent patients who never received ERT or HSCT, and green x marks represent controls. For blood samples, plasma was used for patients and serum for controls.

FIG. 5 shows NT-proCNP levels in plasma from MPS IVA patients and healthy controls.

FIG. 6 shows collagen type I (A) and II (B) levels in patients and controls.

FIG. 7 shows the difference in collagen type I levels between serum and plasma samples. Each dot represents the concentration of collagen type I in plasma (horizontal axis) and serum (vertical axis) collected simultaneously from the same patient or healthy control.

FIG. 8. Shoes collagen type II levels, difference in serum or plasma. Each dot represents the concentration of collagen type II in plasma (horizontal axis) and serum (vertical axis) collected simultaneously from the same patient or healthy control.

### DETAILED DESCRIPTION

Despite progress in the pharmacological management of lysosomal storage disorders, such as the deployment of enzyme replacement therapies (ERT) for Mucopolysaccharidosis Type IVA (MPS IVA), the accurate monitoring and targeted treatment of skeletal growth failure remains a clinical blind spot. Traditional clinical paradigms generally rely on urinary keratan sulfate (KS) to track disease progression; however, this creates a misleading surrogate endpoint because ERT aggressively clears localized renal storage while systemic chondrocyte deterioration can continue undetected. Despite breakthrough progress in the pharmacological management of lysosomal storage disorders, such as the deployment of enzyme replacement therapies (ERT) for Mucopolysaccharidosis Type IVA (MPS IVA), the accurate monitoring and targeted treatment of skeletal growth failure remains a critical clinical blind spot. To overcome this anatomical disconnect and biomarker void, the methods and systems described herein provide for automated clinical decision-support systems (CDSS) and integrated methods of treatment that utilize plasma N-terminal pro-C-type natriuretic peptide (NT-proCNP) as a indicator of skeletal growth failure.

### Identifying Skeletal Growth Failure and Risk

As described herein (e.g., in the Examples), the instant discovery that NT-proCNP is elevated in the plasma of MPS IVA patients and that NT-proCNP concentrations in patients negatively correlates with height z-score provides a foundation for CDSS and CDS methods, and new methods of treatment targeting the underlying C-type natriuretic peptide (CNP) signaling pathway.

NT-proCNP generally refers to and includes the biologically inactive N-terminal propeptide fragment of C-type natriuretic peptide (CNP). In humans, NT-proCNP comprises a 50-amino acid sequence corresponding to amino acids 24 through 73 of the CNP precursor protein identified by UniProt Accession No. P23582. NT-proCNP is generally generated when the 103-amino acid prohormone (proCNP, corresponding to amino acids 24-126 of UniProt P23582) is enzymatically cleaved by furin to yield the biologically active CNP-53 and the highly stable NT-proCNP fragment. Considering that NT-proCNP is co-secreted in an equimolar ratio with active CNP but possesses a substantially longer circulating half-life, NT-proCNP can represent a measurable circulating biomarker of CNP and of systemic chondrocyte activity and longitudinal bone growth. CNP generally refers to and includes C-type natriuretic peptide, a mature, biologically active peptide hormone that generally serves as a primary endogenous regulator of endochondral ossification and longitudinal bone growth. In humans, the active form predominantly exists as CNP-53, a 53-amino acid peptide generated alongside NT-proCNP following the proteolytic cleavage of the precursor proCNP by furin.

Examples of methods for using NT-proCNP in the identification of skeletal growth failure in an individual having Mucopolysaccharidoses (MPS) Type IVA, include methods that comprise detecting the presence of NT-proCNP in plasma and/or measuring the concentration of NT-proCNP in plasma. Non-limiting examples include methods of identifying skeletal growth failure in an individual having Mucopolysaccharidoses (MPS) Type IVA, the methods comprising: obtaining a biological sample from the individual; detecting the presence of N-terminal pro-C-type natriuretic peptide (NT-proCNP) polypeptide and generating a measured concentration of NT-proCNP polypeptide in the biological sample; and identifying the individual as having skeletal growth failure or an increased risk of skeletal growth failure if the measured concentration of NT-proCNP is greater than an age- and sex-matched reference concentration, the age- and sex-matched reference concentration comprising the mean concentration of NT-proCNP polypeptide in age- and sex-matched healthy individuals.

Skeletal growth failure (also referred to as growth impairment) generally refers to and includes the pathological impairment, severe attenuation, or cessation of endochondral ossification, resulting in restricted longitudinal bone development. Clinically, skeletal growth failure can be objectively quantified and longitudinally tracked using a height z-score. Height z-score generally refers to and includes a standardized biometric measurement quantifying the deviation of a patient's longitudinal stature from the mean of a healthy, age- and sex-matched reference cohort (e.g., utilizing standard CDC Growth Charts). As described herein, a negative height z-score can serve as a phenotypic indicator of skeletal growth failure.

Mucopolysaccharidosis Type IVA (MPS IVA), also known as Morquio A syndrome, generally refers to and includes an autosomal recessive lysosomal storage disorder characterized by a systemic deficiency or absence of the enzyme N-acetylgalactosamine-6-sulfatase (GALNS). This enzymatic deficiency generally results in the pathological accumulation of specific glycosaminoglycans (GAGs), primarily keratan sulfate (KS) and chondroitin-6-sulfate (C6S), within lysosomes throughout the body.

Detecting the presence of an NT-proCNP polypeptide can, in certain instances be achieved by any known protein quantification methods. For example, protein detection and quantification methods can include, but not be limited to, affinity-based detection assay. Exemplary affinity-based detection assays include immunoassays. Non-limiting examples of immunoassays include enzyme-linked immunosorbent assays (ELISA), chemiluminescent microparticle immunoassays (CMIA), electrochemiluminescence immunoassays (ECLIA), multiplex bead-based suspension arrays, and surface plasmon resonance (SPR) biosensors, as well as traditional formats such as radioimmunoassays (RIA), Western blotting, and lateral flow assays. As described in the Examples, the immunoassay can be an NT-proCNP ELISA (e.g., Cat #BI-20812, BIOMEDICA, Vienna, Austria).

Age- and sex-matched reference concentration (or reference threshold) generally refers to and includes a biomarker baseline (e.g., mean or median baseline) derived from a normative, healthy control cohort that shares the same sex and corresponding chronological age or age interval as the patient. Age-matched refers to and includes a cohort-based reference group comprising healthy individuals within a defined chronological age or chronological age interval (e.g., an age bracket as exemplified in the Examples) corresponding to the age of a patient. Non-limiting examples of chronological age intervals include, but are not limited to, 0-9 years, 10-19 years, and ≥20 years. Sex-matched refers to and includes a reference group comprising healthy individuals of the same biological sex as a patient. Reference concentrations for age- and sex-matched health reference concentrations can include those described in the Examples, for example (but not limited to), those measured in the Examples data and those described in Olney R C et al. Amino-terminal propeptide of C-type natriuretic peptide (NTproCNP) predicts height velocity in healthy children. Clin Endocrinol (Oxf). 2012 September; 77(3):416-22. doi: 10.1111/j.1365-2265.2012.04392.x. PMID: 22435455; PMCID: PMC3466812.

Examples of instances where NT-proCNP is greater than the age- and sex-matched reference concentration, include but are not limited to, patient NT-proCNP concentrations that are equal to or greater than 1.0 standard deviation (SD) above the age- and sex-matched reference concentration, or equal to or greater than 2.0 standard deviation (SD) above the age- and sex-matched reference concentration.

### Methods of Treatment

As described herein, the identification NT-proCNP provides for CNP as a therapeutic agent against the growth impairment in MPS IVA. Accordingly, contemplated herein are methods of treating skeletal growth failure in an individual having MPS IVA by administering a CNP analogue (e.g., vosoritide or navepegritide) to the individual. For example, such methods include, but are not limited to, (a) measuring a concentration of N-terminal pro-C-type natriuretic peptide (NT-proCNP) in a blood plasma sample obtained from the individual; (b) determining that the concentration of NT-proCNP is greater than an age- and sex-matched reference concentration; and (c) administering to the patient an effective amount of a C-type natriuretic peptide (CNP) analog, the CNP analogue comprising vosoritide or navepegritide, and the CNP analog being administered based on the determination that the measured concentration is greater than the age- and sex-matched reference concentration.

Vosoritide generally refers to and includes the FDA-approved modified recombinant human C-type natriuretic peptide (CNP) analog, or a pharmaceutically acceptable salt thereof, marketed as VOXZOGO®. Vosoritide is generally characterized by an amino acid sequence comprising the 22 C-terminal amino acids of human CNP-22 and a 17-amino acid N-terminal extension, providing increased resistance to neutral endopeptidase (NEP) degradation. Dosing of vosoritide is established according to recognized and FDA approved clinical protocols based on patient-specific metrics, including chronological age and body weight (e.g., approximately 15 ug/kg). Vosoritide is typically administered via a parenteral route, such as by once-daily subcutaneous injection. Navepegritide generally refers to and includes the FDA-approved long-acting C-type natriuretic peptide (CNP) analog, or a pharmaceutically acceptable salt thereof, marketed as YUVIWEL®. Navepegritide is generally characterized by a CNP variant reversibly linked to a biocompatible carrier (e.g., a methoxypolyethylene glycol (mPEG) carrier) through a cleavable linker, designed to provide continuous systemic exposure of active CNP. Dosing of navepegritide is established according to FDA approved and recognized clinical protocols based on patient-specific metrics, including chronological age and body weight (e.g., approximately 100 ug/kg). Navepegritide is typically administered via a parenteral route, such as by once-weekly subcutaneous injection. Generally, the administration of the CNP analogue reduces the plasma concentration of NT-proCNP (or CNP) in the individual.

Examples of instances where NT-proCNP is greater than the age- and sex-matched reference concentration, include but are not limited to, patient NT-proCNP concentrations that are equal to or greater than 1.0 standard deviation (SD) above the age- and sex-matched reference concentration, or equal to or greater than 2.0 standard deviation (SD) above the age- and sex-matched reference concentration.

### CDS Systems and Methods

Provided and described herein are Clinical Decision-Support (CDS) systems and computer-implemented methods configured for the objective, automated detection of skeletal growth failure states in patients with Mucopolysaccharidosis Type IVA (MPS IVA). By integrating hardware-implemented data extraction with specialized clinical logic, the disclosed systems and methods transform biomarker data into clinical alerts. This technical architecture helps identify pathologically elevated NT-proCNP concentrations and normalize against developmental baselines in real-time, and in certain instances, facilitating immediate therapeutic intervention at the point of care.

Non-limiting examples include CDS systems for identifying skeletal growth failure in Mucopolysaccharidosis Type IVA (MPS IVA), the system comprising: (a) a network communication interface configured to obtain laboratory data packets from a clinical data repository for a patient; (b) a processor; and (c) a non-transitory computer-readable memory storing instructions that, when executed by the processor, cause the system to: (i) extract a first digital payload and a second digital payload from the clinical data repository, the first digital payload quantifying a plasma N-terminal pro-C-type natriuretic peptide (NT-proCNP) concentration for the patient, and the second digital payload identifying a chronological age of the patient; (ii) query an age-stratified reference data structure using the second digital payload to retrieve an age- and sex-matched reference concentration; (iii) execute a clinical logic gate that detects a skeletal growth failure state when the plasma NT-proCNP concentration is greater than the age- and sex-matched reference concentration; and (iv) generate a network control signal to trigger an interruptive clinical alert on a clinical endpoint device based on the detected skeletal growth failure state.

### System Architecture and Hardware Foundation

The Clinical Decision-Support (CDS) systems described herein are generally implemented as a specialized computing environment comprising a network communication interface, a processor, and a non-transitory computer-readable memory. The system can be configured as a standalone server, a modular software component integrated within a hospital information system, or a distributed cloud-based architecture. This hardware foundation facilitates the obtainment (e.g., ingesting and/or accessing) of clinical data and its transformation into actionable, real-time interruptive clinical alerts (diagnostic alerts).

The network communication interface generally comprises the hardware and software components (e.g., a network interface controller, wireless transceivers, and/or physical communication ports) necessary to establish a secure, bidirectional data link with an external clinical data repository. The interface can be configured to utilize standard medical data exchange protocol (e.g., including, but not limited to, Health Level Seven (HL7) or Fast Healthcare Interoperability Resources (FHIR)) to obtain laboratory data packets from sources such as Electronic Medical Records (EMR), Laboratory Information Management Systems (LIMS), or cloud-based data warehouses.

The processor generally includes one or more central processing units (CPUs), graphics processing units (GPUs), or application-specific integrated circuits (ASICs) communicatively coupled to the memory and the interface. The processor generally acts as the central logic engine, responsible for (i) the high-speed extraction of digital payloads from incoming data packets and (ii) the execution of the clinical logic gate. By utilizing specialized hardware, the processor can perform continuous, longitudinal monitoring of data streams with minimal latency.

The non-transitory computer-readable memory generally provides persistent storage for the machine-executable instructions governing system operation. For example, the memory can store the age-stratified reference data structure (e.g., a relational or non-relational database) containing the normative NT-proCNP concentration curves. This localized or cached storage generally enables the processor to retrieve age- and sex-matched reference concentrations and execute the logic gate.

In a distributed embodiment, the CDS system generally manages the flow of information across a network between a centralized logic server and remote clinical endpoint devices (e.g., physician workstations, tablets, or mobile devices). In this configuration, the processor generally generates a specialized network control signal designed to traverse the network and trigger an interruptive clinical alert directly on the endpoint device's user interface, ensuring that, for example, a detected skeletal growth failure state is brought to the attention of the clinician at the point of care.

### Clinical Data Integration and Retrieval

The clinical data repository generally serves as the primary data source for the CDS system and methods, and may be implemented as an Electronic Medical Record (EMR), a Laboratory Information Management System (LIMS), or a consolidated hospital data warehouse. The laboratory data packets obtained from the repository generally are structured digital objects (e.g., JSON, XML, or HL7 v2.x messages) that encapsulate the patient's data and/or medical results. These packets can be parsed by the system to extract one or more digital payloads, for example, a “first digital payload” representing the NT-proCNP concentration and a “second digital payload” representing the patient's chronological and sex data, ensuring that biomarker values are accurately synchronized with the patient's age and sex at the time of sample collection.

In certain instances, the network communication interface can be configured to asynchronously ingest the laboratory data packets into the CDS system. This generally allows the CDS system to continuously monitor new biomarker results as they are output by the repository (e.g., without interrupting the clinical workflow or causing latency in the source database).

In certain instances, the network communication interface can also be configured to access the clinical data repository through authenticated handshake protocols. This active access generally allows the interface to query the repository for new entries tagged with specific biomarker codes (e.g., NT-proCNP LOINC codes) or to retrieve historical patient data required for calculating data (e.g., height velocity). By providing the interface with direct access credentials and handshaking capabilities, the CDS system can operate as an automated monitoring agent that functions independently of manual data entry, for example, to ensure that a detected skeletal growth failure state is identified the moment the data becomes available in the network.

### Digital Payload Extraction and Data Parsing

Following the obtaining, accessing, or ingesting of the laboratory data packets from the clinical data repository, the processor can execute instructions to extract a first digital payload and a second digital payload. To isolate these payloads, the processor identifies specific data fields within the packet (e.g., an “Observation” (OBX) segment in an HL7 message or a specific key-value pair in a JSON object). The first digital payload can be extracted as a discrete numerical value quantifying the plasma NT-proCNP concentration. Simultaneously, the second digital payload is extracted to identify both a chronological age and a sex of the patient. This extraction process helps ascertain that the system isolates the specific diagnostic variables required for the logic gate from the broader set of data (e.g., metadata) contained within the obtained data packets.

The extraction logic can further be configured to perform data normalization on the payloads extracted from the obtained packets. For the first digital payload, the processor identifies the units of measure (e.g., pmol/L or pg/mL) and, if necessary, applies a conversion factor to align the value with the scale used in the reference data structure. For the second digital payload, the processor can be configured to calculate the chronological age in real-time if the obtained data packet provides a Date of Birth (DOB) instead of a discrete age. For example, this calculation is performed relative to the timestamp of the NT-proCNP sample collection to ensure accuracy to the month or day. Furthermore, the processor can standardize the sex payload into a format compatible with an age-stratified reference data structure. By normalizing these payloads, the system can ensure that the subsequent logic gate execution is based on high-fidelity, standardized inputs regardless of the source or format of the clinical data repository.

### Age-Stratified Reference Data Structure

The age-stratified reference data structure is stored within the non-transitory memory and can be configured as a searchable digital architecture, such as a relational database or a look-up table. The structure can generally be stratified by organizing normative NT-proCNP concentration values into discrete chronological ages or age intervals (e.g., monthly or yearly increments, or broader brackets such as 0-9 years, 10-19 years, and ≥20 years) and further sub-divided by biological sex. These strata can contain statistically derived reference concentrations, such as mean values, 95th percentiles, or specific standard deviation thresholds (e.g., ≥1 SD or ≥2 SD) established from healthy control cohorts (e.g., described herein and in Olney et al.).

The processor can be configured to query the age-stratified reference data structure using the second digital payload as a primary query key. By matching the extracted age and sex data of the patient to the corresponding stratum within the data structure, the processor can retrieve the specific age- and sex-matched reference concentration required for the clinical logic gate. This architecture can ensure that the system normalizes the patient's NT-proCNP concentration against the correct developmental baseline (e.g., age or age interval).

### Clinical Logic Gate and Predictive Detection

Generally, the processor can be configured to execute a clinical logic gate that performs a comparative analysis between the first digital payload (the patient's NT-proCNP concentration) and the retrieved age- and sex-matched reference concentration. For example, the logic gate detects a skeletal growth failure state when the plasma NT-proCNP concentration is determined to be greater than the reference concentration. This “greater than” condition may be satisfied by a direct numerical comparison or by a determination that the concentration exceeds a predefined statistical threshold, such as the 95th percentile or a value≥1.0 SD or ≥2.0 SD above the normative mean for the patient's age and sex.

The execution of the logic gate can, in certain instances, represent a technical transformation of the raw digital payloads into a diagnostic status. For example, by normalizing the patient's biomarker data against the correct developmental baseline retrieved from the reference data structure, the logic gate can output pathological biomarker elevations associated with MPS IVA. Upon the detection of the skeletal growth failure state, the logic gate can trigger the subsequent generation of a network control signal, such that the detected failure state is translated into an actionable clinical intervention.

### Network Signal Generation and Clinical Alerting

Based on the detected skeletal growth failure state, the processor can generate a network control signal to trigger an interruptive clinical alert on a clinical endpoint device. This network control signal is generally an instruction-bearing digital payload configured to traverse the network communication interface via asynchronous transmission protocols. By utilizing an asynchronous network control signal, the system can ensure that the alert is delivered without blocking other critical data traffic within the clinical environment, allowing the processor to continue monitoring subsequent patient data packets while the alert is being propagated to the clinician.

In certain instances, the network control signal can be configured to modify a physical data structure within the clinical data repository. For example, the processor can generate and transmit an application programming interface (API) request to the repository (e.g., an EMR or LIMS) to persistently store a classification tag in the patient's electronic medical record. This classification tag generally serves as a digital marker (e.g., permanent digital marker), such as a “Skeletal Growth Failure” or “Growth Impairment” flag, that is automatically appended to the patient's metadata. This modification of the physical data structure can ensure that the detected growth failure state remains associated with the patient's record for longitudinal tracking and auditability, even after the initial interruptive alert has been acknowledged.

In certain instances, the network control signal can be configured to modify the rendering instructions of a graphical user interface (GUI) on the clinical endpoint device. For example, rather than sending a text message, the signal provides specific machine-executable instructions that cause the GUI to overlay the interruptive clinical alert directly on top of the active electronic medical record view. This overlay can be rendered as a primary-focus element (e.g., a modal window or a high-contrast banner) that overrides standard display priorities. By modifying the GUI rendering instructions in real-time, the system can ensure that the alert is visually noticeable (e.g., forcing a clinical “stop point” that prompts the user to consider therapeutic intervention, such as the administration of a CNP analog).

### Computational Methods and Non-Transitory Computer-Readable Media

Also described and provided herein are computer-implemented methods for detecting skeletal growth failure states, representing the algorithmic process steps executed by the system's processor. These methods are embodied in machine-readable instructions stored on non-transitory computer-readable media to facilitate the automated, real-time diagnostic monitoring and network alerting functions described herein.

### ADDITIONAL DEFINITIONS

An “individual” or “subject” are interchangeable and generally refers to and includes any human or non-human animal to whom a composition or method can be administered, including humans, non-human primates, and other mammals.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “up to,” “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

In some instances, a value is “about” a recited value if the value is within ±10% of the recited value. In some instances, disclosure of “about” a recited value includes disclosure of a value that is within ±5% of the recited value.

The term “comprise” and variations thereof, such as, “comprises” and “comprising” are to be construed in an open, inclusive sense, that is, as “including, but not limited to.” It is also to be understood that disclosure of “comprising” is also intended to include disclosures of “consisting essentially of” and “consisting of” the same recited elements. Generally, “consisting essentially of” is to construed at partially open, being directed to those elements set forth and to those other elements that do not materially affect the basic and novel characteristics of the invention described. Generally, “consisting of” means that the claim is directed only to the specified elements.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each were set out individually herein.

### EXEMPLARY EMBODIMENTS

In addition to those already described herein, non-limiting examples of embodiments provided and described herein also include, but are not limited to, the following enumerated embodiments:

Embodiment 1: A method of identifying skeletal growth failure in an individual having Mucopolysaccharidoses (MPS) Type IVA, the method comprising: obtaining a biological sample from the individual; detecting the presence of N-terminal pro-C-type natriuretic peptide (NT-proCNP) polypeptide and generating a measured concentration of NT-proCNP polypeptide in the biological sample; and identifying the individual as having skeletal growth failure or an increased risk of skeletal growth failure if the measured concentration of NT-proCNP is greater than an age- and sex-matched reference concentration, the age- and sex-matched reference concentration comprising the mean concentration of NT-proCNP polypeptide in age- and sex-matched healthy individuals.

Embodiment 2: The method of embodiment 1, wherein the age of the individual is 8 years or greater.

Embodiment 3: The method of embodiment 1 or embodiment 2, wherein the biological sample is a blood plasma sample.

Embodiment 4: The method of any one of embodiments 1-3, wherein the presence of N-terminal pro-C-type natriuretic peptide (NT-proCNP) polypeptide is detected by an affinity-based detection assay.

Embodiment 5: The method of embodiment 4, wherein the affinity-based detection assay is an ELISA.

Embodiment 6: The method of any one of embodiments 1-5, wherein the individual is identified as having skeletal growth failure or increased risk of skeletal growth failure if the measured concentration the measured concentration of NT-proCNP is equal to or greater than 2.0 standard deviations above the reference concentration.

Embodiment 7: A method of treating skeletal growth failure in an individual having MPS IVA, the method comprising: measuring a concentration of N-terminal pro-C-type natriuretic peptide (NT-proCNP) in a blood plasma sample obtained from the individual; determining that the concentration of NT-proCNP is equal to or greater than 1.0 standard deviations above an age- and sex-matched reference concentration; and administering to the patient an effective amount of a C-type natriuretic peptide (CNP) analog, the CNP analogue comprising vosoritide or navepegritide, and the CNP analog being administered based on the determination that the measured NT-proCNP concentration is equal to or greater than the 1.0 standard deviation above the age- and sex-matched reference concentration.

Embodiment 8: The method of embodiment 7, wherein the measured concentration of NT-proCNP is equal to or greater than 2.0 standard deviations above an age- and sex-matched reference concentration.

Embodiment 9: The method of embodiment 7 or embodiment 8, wherein concentration of N-terminal pro-C-type natriuretic peptide (NT-proCNP) polypeptide is measured by an affinity-based detection assay.

Embodiment 10: The method of embodiment 9, wherein the affinity-based detection assay is an ELISA.

Embodiment 11: The method of any one of embodiments 7-10, wherein the individual has received and/or is concurrently receiving elosulfase alfa.

Embodiment 12: The method of any one of embodiments 7-11, wherein the CNP analogue reduces the concentration of NT-proCNP in the individual.

Embodiment 13: A clinical decision-support (CDS) system for identifying skeletal growth failure in Mucopolysaccharidosis Type IVA (MPS IVA), the system comprising: (a) a network communication interface configured to obtain laboratory data packets from a clinical data repository for a patient; (b) a processor; and (c) a non-transitory computer-readable memory storing instructions that, when executed by the processor, cause the system to: (i) extract a first digital payload and a second digital payload from the clinical data repository, the first digital payload quantifying a plasma N-terminal pro-C-type natriuretic peptide (NT-proCNP) concentration for the patient, and the second digital payload identifying a chronological age of the patient; (ii) query an age-stratified reference data structure using the second digital payload to retrieve an age- and sex-matched reference concentration; (iii) execute a clinical logic gate that detects a skeletal growth failure state when the plasma NT-proCNP concentration is greater than the age- and sex-matched reference concentration; and (iv) generate a network control signal to trigger an interruptive clinical alert on a clinical endpoint device based on the detected skeletal growth failure state.

Embodiment 14: A computer-implemented method for identifying skeletal growth failure in Mucopolysaccharidosis Type IVA (MPS IVA), the method comprising: (a) obtaining, by a processor via a network communication interface, laboratory data packets from a clinical data repository; (b) extracting, by the processor, a first digital payload and a second digital payload from the laboratory data packets, the first digital payload quantifying a plasma N-terminal pro-C-type natriuretic peptide (NT-proCNP) concentration for a patient, and the second digital payload identifying a chronological age of the patient; (c) querying, by the processor, an age-stratified reference data structure using the second digital payload to retrieve an age- and sex-matched concentration; (d) executing, by the processor, a clinical logic gate that detects a skeletal growth failure state when the plasma NT-proCNP concentration is greater than the age- and sex-matched concentration; and (e) generating, by the processor, a network control signal to trigger an interruptive clinical alert on a clinical endpoint device based on the detected skeletal growth failure state.

Embodiment 15: A non-transitory computer-readable medium storing instructions that, when executed by a processor of a clinical decision-support (CDS) system, cause the system to perform operations for identifying skeletal growth failure in Mucopolysaccharidosis Type IVA (MPS IVA), the operations comprising: (a) obtaining laboratory data packets from a clinical data repository; (b) extracting a first digital payload and a second digital payload from the laboratory data packets, the first digital payload quantifying a plasma N-terminal pro-C-type natriuretic peptide (NT-proCNP) concentration for a patient, and the second digital payload identifying a chronological age of the patient; (c) querying an age-stratified reference data structure using the second digital payload to retrieve an age- and sex-matched reference threshold; (d) executing a clinical logic gate detects a skeletal growth failure state when the plasma NT-proCNP concentration is greater than the age- and sex-matched reference threshold; and (e) generating a network control signal to trigger an interruptive clinical alert on a clinical endpoint device based on the detected skeletal growth failure state.

Embodiment 16: The system or method or non-transitory computer-readable medium of any one of embodiments 13-15, wherein the clinical logic gate detects a skeletal growth failure state when the plasma NT-proCNP concentration is equal to or greater than 1.0 standard deviation or 2.0 standard deviations above the age- and sex-matched reference concentration.

Embodiment 17: The system or method or non-transitory computer-readable medium of any one of embodiments 13-16, wherein the network communication interface is configured to asynchronously ingest the laboratory data packets into the clinical data repository.

Embodiment 18: The system or method or non-transitory computer-readable medium of embodiment 17, wherein the network control signal modifies a physical data structure within the clinical data repository by automatically appending a classification tag to an electronic medical record of the patient, and transmitting an asynchronous network control signal to a clinical endpoint device to render the interruptive clinical alert.

Embodiment 19: The system or method or non-transitory computer-readable medium of any one of embodiments 13-16, wherein the network communication interface is configured to access the clinical data repository.

Embodiment 20: The system or method or non-transitory computer-readable medium of embodiment 19, wherein the network control signal is configured to modify rendering instructions of a graphical user interface (GUI) to overlay the interruptive clinical alert on an electronic medical record.

Embodiment 21: The system or method or non-transitory computer-readable medium of embodiment 19, wherein the instructions further cause the system to transmit an application programming interface (API) request to persistently store a classification tag in the clinical data repository.

Embodiment 22: The system or method or non-transitory computer-readable medium of any one of embodiments 13-21, wherein the clinical data repository comprises a backend database server of an electronic health record (EHR) system, a laboratory information system (LIS), or a health information exchange (HIE).

Embodiment 23: The system or method or non-transitory computer-readable medium of any one of embodiments 13-22, wherein the age- and sex-matched reference concentration comprises the mean concentration of NT-proCNP polypeptide in age- and sex-matched healthy individuals.

Embodiment 24: A clinical decision-support (CDS) system for identifying skeletal growth failure in Mucopolysaccharidosis Type IVA (MPS IVA) and generating an interruptive clinical alert, the system comprising: (a) a network communication interface configured to asynchronously ingest laboratory data packets into a clinical data repository for a patient; (b) a processor; and (c) a non-transitory computer-readable memory storing instructions that, when executed by the processor, cause the system to: (i) extract a first digital payload and a second digital payload from the clinical data repository, the first digital payload quantifying a plasma N-terminal pro-C-type natriuretic peptide (NT-proCNP) concentration for the patient, and the second digital payload identifying a chronological age and sex of the patient; (ii) query an age- and sex-stratified reference data structure stored in the memory using the second digital payload to retrieve a dynamic age- and sex-matched baseline threshold; (iii) execute a clinical logic gate detects a skeletal growth failure state when the plasma NT-proCNP concentration quantified in the first digital payload is equal to or greater than 2.0 standard deviations above the dynamic age- and sex-matched baseline threshold; and (iv) modify a physical data structure within the clinical data repository by automatically appending a classification tag to an electronic medical record of the patient, and transmitting an asynchronous network control signal to a clinical endpoint device to render the interruptive clinical alert.

Embodiment 25: A clinical decision-support (CDS) system for identifying skeletal growth failure in Mucopolysaccharidosis Type IVA (MPS IVA) and generating an interruptive clinical alert, the system comprising: (a) a network communication interface configured to access a clinical data repository; (b) a processor; and (c) a non-transitory computer-readable memory storing instructions that, when executed by the processor, cause the system to: (i) retrieve a first digital payload and a second digital payload from the clinical data repository, the first digital payload quantifying a plasma N-terminal pro-C-type natriuretic peptide (NT-proCNP) concentration for a patient, and the second digital payload identifying a chronological age of the patient; (ii) execute a clinical logic gate in volatile memory during runtime to detect a skeletal growth failure state by comparing the first digital payload against an age- and sex-matched reference threshold determined by the second digital payload, wherein the skeletal growth failure state is detected when the plasma NT-proCNP concentration is equal to or greater than 2.0 standard deviations above the age- and sex-matched reference threshold; and (iii) modify rendering instructions sent to a clinical endpoint device to overlay the interruptive clinical alert on a graphical user interface (GUI) displaying an electronic medical record of the patient.

### EXAMPLES

### Example 1

### 1.1.1 Summary

Mucopolysaccharidosis type IVA (MPS IVA, Morquio A syndrome) is a rare inherited disorder characterized by skeletal dysplasia due to deficient N-acetylgalactosamine-6-sulfate sulfatase activity, resulting in glycosaminoglycan (GAG) accumulation. Identifying accurate biomarkers reflecting clinical severity and therapeutic response remains challenging.

Characterization of GAGs in urine, blood, and cerebrospinal fluid has been performed for several decades. Previous studies indicated no correlation between urine and serum GAG levels in MPS II and MPS IVA patients. Blood and urine KS levels follow different age-dependent patterns and sulfation levels. Another report regarding MPS I patients suggested that the oligosaccharide storage pattern (the ratio of heparan sulfate to dermatan sulfate) in urine reflects the storage in the kidney, which is different from the storage pattern in serum, liver, and brain. These findings suggest that urinary GAG is derived primarily from GAGs stored or reabsorbed in the kidney. Some urinary GAG may be derived from small GAG fragments (oligosaccharides rather than a large saccharide chain) that filter through the kidneys from the circulation. Thus, blood GAGs would more closely reflect GAGs stored in the whole body and may be a more informative indicator of systemic disease burden than urinary GAGs.

In MPS IVA, blood and urinary KS levels correlate to some extent with clinical severity in the early and progressive stages of the disease, before the growth plate is destroyed, since KS synthesis decreases rapidly after the teenage years, and KS levels in MPS IVA patients are naturally normalized or subnormal by adulthood. Therefore, KS can only be useful as a biomarker before the teenage years. In addition, even before the teenage years, there is a lack of reliable biomarkers that correlate closely with skeletal symptoms.

Blood KS in MPS IVA directly indicates growth and/or repair of the cartilage, where it is mainly synthesized. Most blood KS is derived from chondrocytes and their extracellular matrix; therefore, its reduction would be a more direct indicator of bone improvement. However, there is an overlap in blood KS levels between patients and age-matched controls. On the other hand, urinary KS can distinguish more MPS IVA patients from age-matched controls than blood KS; however, there is no correlation between urinary KS reduction and clinical improvement of skeletal lesions during enzyme replacement therapy (ERT). After starting ERT, most patients show a decrease in urinary KS levels because the enzyme is delivered to the kidney and digests KS stored in the kidney, but a concomitant decrease in blood KS levels has not yet been reported because urinary KS does not reflect blood KS coming directly from bone and cartilage. Thus, urinary KS is functional in demonstrating the pharmacodynamic effects of therapy, but it does not provide a valuable surrogate biomarker of skeletal or clinical improvement during these therapies for MPS IVA. In summary, blood KS reflects overall skeletal symptoms more accurately than urinary KS, particularly in patients undergoing ERT. However, blood KS is less sensitive than urinary KS when diagnosing patients. Conversely, urinary KS presents a challenge in effectively monitoring therapeutic response in patients receiving ERT.

Due to the current limitations described above, developing novel biomarkers remains an unmet challenge, prompting researchers to explore alternative biomarkers that can more accurately reflect the skeletal pathology of MPS IVA. N-terminal pro-C-type natriuretic peptide (NT-proCNP), collagen type I, and collagen type II have been investigated as candidate biomarkers in human blood because of their importance in the skeletal system, particularly in the epiphyseal plate, connective tissues, and cartilage, respectively. Additionally, two types of KS, mono-sulfated KS (Galβ1→4GlcNAc(6S)) and di-sulfated KS (Galβ1(6S)→4GlcNAc(6S)), have been separately evaluated to elucidate their respective significance.

Potential surrogate biomarkers, including NT-proCNP, collagen types I and II, mono-sulfated keratan sulfate (KS), di-sulfated KS, and chondroitin-6-sulfate (C6S), have been evaluated in blood and urine samples from patients ranging from childhood to adulthood. NT-proCNP levels are significantly elevated in patients of all ages and negatively correlated with growth impairment, especially after childhood. Collagen type I levels increase significantly in adult patients, whereas collagen type II levels show age-dependent elevations. Urinary KS, both mono- and di-sulfated forms, demonstrate moderate negative correlations with growth impairment. Moreover, NT-proCNP, mono- and di-sulfated KS in plasma, and urinary di-sulfated KS are not affected by enzyme replacement therapy in younger patients, unlike urinary mono-sulfated KS.

NT-proCNP is significantly elevated in the plasma of MPS IVA patients in all comparable age groups. In addition, the concentrations in patients negatively correlate with height z-score, the most reliable indicator of skeletal growth failure.

In conclusion, NT-proCNP has emerged as an independent biomarker that reflects the severity of skeletal dysplasia and may also predict future growth rates. These findings highlight the potential role of NT-proCNP in clinical assessment and monitoring therapeutic efficacy, addressing current unmet needs in MPS IVA management.

### 1.1.2 Abstract

Mucopolysaccharidosis type IVA (MPS IVA, Morquio A syndrome) is a rare inherited disorder characterized by skeletal dysplasia due to deficient N-acetylgalactosamine-6-sulfate sulfatase activity, resulting in glycosaminoglycan (GAG) accumulation. Identifying accurate biomarkers reflecting clinical severity and therapeutic response remains challenging. This study evaluated potential surrogate biomarkers, including N-terminal pro-C-type natriuretic peptide (NT-proCNP), collagen types I and II, mono-sulfated keratan sulfate (KS), di-sulfated KS, and chondroitin-6-sulfate (C6S), in blood and urine samples from patients across a broad age range. NT-proCNP levels were significantly elevated in patients of all ages and negatively correlated with growth impairment, especially after childhood. Collagen type I levels significantly increased in adult patients, whereas collagen type II showed age-dependent elevations. Urinary KS, mono- and di-sulfated forms, demonstrated moderate negative correlations with growth impairment. Moreover, NT-proCNP, mono- and di-sulfated KS in plasma, and urinary di-sulfated KS were not affected by enzyme replacement therapy in younger patients, unlike urinary mono-sulfated KS. In conclusion, NT-proCNP has emerged as a promising independent biomarker reflecting the severity of skeletal dysplasia and possibly the future growth rate. These findings highlight the potential role of NT-proCNP in clinical assessment and monitoring therapeutic efficacy, addressing current unmet needs in MPS IVA management.

### 1.1.3 Introduction

Mucopolysaccharidoses (MPS) are a group of inherited lysosomal storage disorders caused by deficiencies in enzymes required to degrade glycosaminoglycans (GAGs), which are integral to connective tissue. The inability to break down GAGs results in accumulation in multiple tissues and organs, leading to systemic dysfunction. Depending on the specific enzyme deficiency, MPS are classified into several subtypes. In some instances, a phenomenon known as pseudodeficiency can potentially complicate diagnosis and prognosis, highlighting the importance of biomarkers that accurately reflect the stage and prognosis of the disease.

Among these, MPS IVA (Morquio A syndrome) is characterized by a deficiency in N-acetylgalactosamine-6-sulfate sulfatase (GALNS) enzyme, which leads to the accumulation of keratan sulfate (KS) and chondroitin-6-sulfate (C6S). MPS IVA is a rare genetic disorder that causes growth impairment and skeletal abnormalities with an estimated incidence that varies by region. The resultant skeletal dysplasia is primarily driven by the accumulation of GAGs (KS and C6S), which disrupts the function of chondrocytes in the growth plates, impairs cartilage matrix development, and inhibits bone matrix mineralization. The severity of bone and cartilage damage differs by the severity of the phenotype (attenuated or severe) and the type of bone affected. Bone growth by endochondral ossification is affected more severely than other types of MPS. Thus, it is a type of skeletal dysplasia affecting bone and cartilage growth. The hallmark feature of MPS IVA is progressive and characterized by incomplete or defective endochondral ossification and a successive imbalance of growth. The skeletal-related symptoms of MPS IVA include prominent forehead, abnormal face with a large mandible, disproportionate short-trunk dwarfism with short neck, cervical spine instability with odontoid hypoplasia, cervical spinal cord compression, pectus carinatum, tracheal deviation and obstruction, restrictive lung, flaring of the rib cage, kyphoscoliosis, platyspondyly, hip dysplasia with coxa valga, genu valgum, hypermobile joints, waddling gait, and pes planus. The degree of imbalance in growth between bone and other organs and tissues contributes to the unique skeletal dysplasia and clinical severity, including the narrowing of the trachea. Respiratory failure has been shown as the primary cause of death in patients, followed by cardiac failure, post-traumatic organ failure, complications of surgery, and myocardial infarction.

In addition to its role in the cardiovascular and neurological systems, C-type natriuretic peptide (CNP) plays a critical role in skeletal development. CNP is synthesized in various tissues, including cartilage, and acts through the natriuretic peptide receptor B (NPR-B) on chondrocytes to activate the receptor's guanylyl cyclase domain, increasing intracellular cGMP levels. Elevated cGMP then activates protein kinase G (PKG), which phosphorylates various proteins controlling gene expression and metabolic processes. This phosphorylation influences transcription factors and other regulators, promoting chondrocyte maturation, extracellular matrix production, and balanced cell proliferation. Through these coordinated intracellular events, CNP ensures the proper progression of chondrocyte differentiation and healthy growth plate development. As a result, the skeletal structure can expand and mature, underscoring CNP's critical role in maintaining the equilibrium between chondrocyte growth and its progression to a fully differentiated state. These processes are crucial for endochondral ossification in the growth plates, facilitating longitudinal bone growth. NT-proCNP, consisting of amino acids, is the biologically inactive cleavage product of proCNP and is secreted in equimolar amounts with CNP. Based on the higher concentration of NT-proCNP in blood than that of CNP, NT-proCNP is thought to have a longer half-life than CNP in circulation, reflecting the systemic expression level of CNP better than CNP itself.

Collagen type I is the most abundant collagen in the human body, forming a critical structural component of bone, skin, tendons, and other connective tissues. It provides the scaffold for bone mineralization and contributes to the strength and rigidity of the skeletal system. Interestingly, studies have shown that the expression of mRNA encoding collagen type I increased in chondrocytes derived from MPS IVA patients.

Collagen type II, on the other hand, is predominantly found in cartilage and serves as a marker for chondrocyte activity, especially in the proliferative zone in growth plates. It is essential for maintaining the tensile strength and structural integrity of cartilage, including the growth plate. As a critical component of the extracellular matrix, collagen type II supports the mechanical properties of cartilage and facilitates chondrocyte differentiation and matrix organization.

This investigation focused on these three biomarkers (NT-proCNP, collagen type I, and collagen type II) to elucidate their relationship with the skeletal manifestations of MPS IVA. By investigating these biomarkers with GAGs (C6S and KS) levels and genotyping, the aim was to identify a novel biomarker that can more accurately predict clinical severity, disease prognosis, and therapeutic efficacy relevant to improvements in bone lesions. These findings have provided valuable insights into the pathophysiology of MPS IVA and informed the development of potential surrogate biomarkers.

### 1.2.1 Subject Characteristics (Age, Sex, Race, Height)

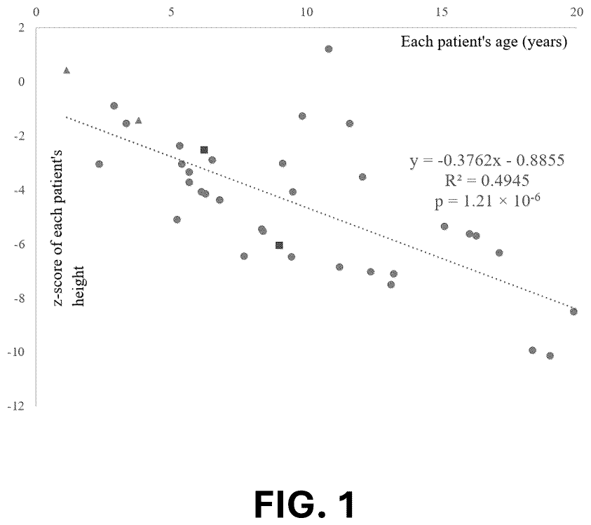

Sixty patients successfully provided blood and/or urine specimens. The characteristics of all participants are summarized in Table 1. Thirty-three patients (55%) were female, and 27 (45%) were male. Forty-four (73.3%) of the participants were White or Caucasian, 5 (8.3%) were Black or African American, 3 (5%) were South Asian, 2 (3.3%) were Southeast Asian, 2 (3.3%) were East Asian, and 4 (6.7%) were other/mixed race. The z-score of each patient's height showed a significant negative correlation with age, indicating that current therapy is not effective enough to prevent growth impairment in MPS IVA (r=−0.703, p=1.21×10\hat{ }-6, FIG. 1).

TABLE 1
Characteristics of all participants

| | | | Height | Age | Genotype | | |
|---|---|---|---|---|---|---|---|
| ID | Sex | Race | (Z-score) | (year) | Allele 1 | Allele 2 | Treatment |
| M01 | F | White or | −6.34 | 17.16 | c.740G > A (G247D) | c.901G > T (G301C) | ERT |
| | | Caucasian | −6.73 | 18.70 | | | |
| M02 | F | Chinese | | 38.57 | c.953T > G (M318R) | c.1567T > G | ERT |
| | | | | 40.00 | | (X523EextX93) | |
| M03 | F | White or | | 24.62 | c.448delC (H150Tfs*3) | c.651_652insG | ERT |
| | | Caucasian | | | | (K218Efs*45) | |
| M04 | F | White or | | 42.12 | c.675dupC | unknown | ERT |
| | | Caucasian | | | (F226Lfs*37) | | |
| | | | | 43.51 | | | |
| M05 | F | White or | −9.93 | 18.35 | c.346G > A (G116S) | c.1156C > T (R386C) | ERT |
| | | Caucasian | −9.76 | 19.78 | | | |
| M06 | F | White or | −8.52 | 19.89 | c.346G > A (G116S) | c.1156C > T (R386C) | ERT |
| | | Caucasian | | 21.29 | | | |
| M07 | M | White or | | 32.25 | c.337A > T (1113F) | c.1171A > G (M391V) | No ERT for 2 years and 8 |
| | | Caucasian | | | | | months |
| M08 | M | White or | No record | 8.00 | c.122T > A (M41K) | c.122T > A (M41K) | ERT |
| | | Caucasian | −6.09 | 9.03 | | | Received HSCT 1 year ago |
| M10 | M | White or | 1.22 | 10.82 | c.338T > C (1113T) | c.1219A > C (N407H) | ERT |
| | | Caucasian | 1.01 | 12.37 | | | |
| M11 | F | White or | −5.36 | 15.11 | c.421T > A (W141R) | unknown | ERT |
| | | Caucasian | −5.53 | 16.55 | | | |
| M12 | F | White or | | 26.65 | c.697G > A (D233N) | c.1034T > C (L345P) | No ERT for 4 years and 4 |
| | | Caucasian | | 28.15 | | | months |
| M13 | M | Black or African | −7.52 | 13.13 | c.251C > A (A84E) | c.319G > A (A107T) | ERT |
| | | American | −6.75 | 14.84 | | | |
| M14 | F | White or | −5.7 | 16.29 | c.740G > A (G247D) | unknown | ERT |
| | | Caucasian | −5.7 | 17.83 | | | |
| M15 | M | White or | −2.39 | 5.32 | c.498delC (F167Lfs*32) | c.901G > T (G301C) | ERT |
| | | Caucasian | −3.84 | 6.81 | | | |
| M16 | M | Filipino | −5.09 | 5.21 | c.228C > A (N76K) | c.1480A > G (p.M494V) | ERT |
| | | | No record | 6.77 | | | |
| M19 | F | Black or African | −6.49 | 9.44 | c.245C > T (S82L) | unknown | ERT |
| | | American | −5.64 | 10.96 | | | |
| M20 | F | White or | −4.09 | 9.49 | c.498delC (F167Lfs*32) | c.1474G > A (A492T) | ERT |
| | | Caucasian | −3.53 | 10.06 | | | |
| M21 | M | White or | −4.08 | 6.12 | c.651_652insG | c.1159G > A (G387S) | ERT |
| | | Caucasian | −5.22 | 7.55 | (K218Efs*45) | | |
| M22 | M | White or | −5.45 | 8.34 | c.651_652insG | c.1159G > A (G387S) | ERT |
| | | Caucasian | −7.24 | 9.78 | (K218Efs*45) | | |
| M25 | M | White or | | 41.34 | c.155C > T (P52L) | c.337A > T (1113F) | Never received ERT |
| | | Caucasian | | 43.28 | | | |
| M26 | M | Filipino | −7.03 | 12.39 | c.93delC (N32Tfs*97) | c.946G > A (G316R) | ERT |
| | | | No record | 13.89 | | | |
| M28 | F | Mixed | | 31.20 | c.868G > A (G290S) | c.868G > A (G290S) | Never received ERT |
| | | Japanese and | | 32.75 | | | |
| | | Caucasian | | | | | |
| M30 | F | White or | | 43.52 | c.121 − 12T > C | c.121 − 12T > C | ERT |
| | | Caucasian | | | | | |
| M31 | M | Black or African | −1.29 | 9.86 | c.935C > G (T312S) | c.1520G > T (C507F) | ERT |
| | | American | −1.11 | 11.44 | | | |
| M32 | F | White or | | 45.50 | c.121 − 12T > C | c.121 − 12T > C | Never received ERT |
| | | Caucasian | | 47.03 | | | |
| M33 | F | White or | | 29.94 | c.1012C > T (Q338X) | c.1171A > G (M391V) | Never received ERT |
| | | Caucasian | | 31.48 | | | |
| M34 | M | White or | | 28.17 | c.121A > T (M41L) | c.121A > T (M41L) | ERT |
| | | Caucasian | | 29.69 | | | |
| M35 | M | White or | −6.86 | 11.23 | c.139G > A (G47R) | c.1156C > T (R386C) | No ERT for 4 years |
| | | Caucasian | −7.69 | 12.64 | | | |
| M36 | F | Asian Indian | | 25.72 | c.346G > A (G116S) | c.346G > A (G116S) | ERT |
| | | | | 27.27 | | | |
| M37 | M | Asian Indian | −10.12 | 19.01 | c.346G > A (G116S) | c.346G > A (G116S) | ERT |
| | | | | 20.56 | | | |
| M38 | F | White or | −5.63 | 16.04 | c.1559G > A (W520X) | unknown | ERT |
| | | Caucasian | | | | | |
| M39 | F | White or | −3.54 | 12.08 | c.1559G > A (W520X) | unknown | ERT |
| | | Caucasian | | | | | |
| M41 | M | White or | | 35.29 | c.121A > T (M41L) | c.498deIC (F167Lfs*32) | ERT |
| | | Caucasian | | 36.86 | | | |
| M42 | F | White or | | 28.24 | c.860C > T (S287L) | c.1055T > C (L358P) | ERT |
| | | Caucasian | | 29.95 | | | |
| M43 | M | White or | | 29.04 | c.121A > T (M41L) | c.901G > T (G301C) | ERT |
| | | Caucasian | | 30.52 | | | |
| M44 | F | White or | | 24.28 | c.1171A > G (p.M391V) | c.502G > A (p.G168R) | ERT |
| | | Caucasian | | 25.85 | | | |
| M45 | F | White or | | 61.08 | c.740G > A (G247D) | c.761A > G (Y254C) | Never received ERT |
| | | Caucasian | | 62.66 | | | |
| M46 | F | White or | No record | 6.92 | c.167C > A (T56N) | c.502G > A (G168R) | ERT |
| | | Caucasian | −5.54 | 8.40 | | | |
| M47 | F | Some other race | | 24.33 | c.139G > A (G47R) | c.466T > C (F156L) | ERT |
| | | | | 25.78 | | | |
| M48 | F | White or | −7.11 | 13.23 | c.139G > A (G47R) | c.466T > C (F156L) | ERT |
| | | Caucasian | −7.66 | 14.68 | | | |
| M49 | M | White or | −6.44 | 7.68 | c.1156C > T (R386C) | c.1156C > T (R386C) | ERT |
| | | Caucasian | | | | | |
| M50 | F | White or | −3.33 | 5.65 | c.451C > A (P151T) | c.477G > A (W159X) | ERT |
| | | Caucasian | −4.81 | 7.26 | | | |
| M51 | M | White or | −0.9 | 2.89 | c.451C > A (P151T) | c.477G > A (W159X) | ERT |
| | | Caucasian | −0.52 | 4.50 | | | |
| M52 | F | Multiple Races | No record | 2.50 | c.651_652insG | unknown | Received HSCT |
| | | | No record | 4.60 | (K218Efs*45) | | 11 months ago |
| | | | −2.54 | 6.21 | | | |
| M53 | F | Chinese | | 23.20 | c.1482 + 5G > C | c.1498G > T (G500C) | No ERT for 13 months |
| | | | | 24.37 | | | ERT |
| M54 | F | White or | | 36.26 | not tested | not tested | No ERT for 5 years |
| | | Caucasian | | | | | and 9 months |
| M55 | F | White or | −3.04 | 5.39 | c.1339G > C (D447H) | unknown | ERT |
| | | Caucasian | −4.2 | 6.80 | | | |
| M56 | F | White or | −1.44 | 3.79 | c.740G > A (G247D) | c.1451C > A (P484H) | Never received ERT |
| | | Caucasian | | | | | |
| M57 | M | White or | 0.41 | 1.13 | c.740G > A(G247D) | c.1451C > A (P484H) | Never received ERT |
| | | Caucasian | | | | | |
| M58 | M | White or | −4.38 | 6.78 | c.1012C > T (Q338X) | c.181C > G (R61G) | ERT |
| | | Caucasian | | | | | |
| M59 | M | White or | −4.14 | 6.26 | c.337A > T (p.1113F) | c.901G > T (p.G301C) | ERT |
| | | Caucasian | | | | | |
| M60 | F | Some other race | | 26.58 | c.901G > T (G301C) | c.1156C > T (R386C) | Never received ERT |
| M63 | F | White or | | 22.72 | c.331C > T (Q111X) | c.1365 − 2A > G | ERT |
| | | Caucasian | | | | | |
| M64 | M | Black or African | −3.74 | 5.64 | c.633 + 1G > C | c.1558T > C (W520R) | ERT |
| | | American | | | | | |
| M65 | M | Black or African | −2.9 | 6.50 | c.633 + 1G > C | c.1558T > C (W520R) | ERT |
| | | American | | | | | |
| M66 | F | White or | −1.56 | 3.34 | c.1156C > T (R386C) | unknown | ERT |
| | | Caucasian | | | | | |
| M67 | M | White or | −1.55 | 11.58 | c.1171 A > G (M391V) | c.331C > T (Q111X) | ERT |
| | | Caucasian | | | | | |
| M68 | M | White or | | 45.78 | c.1156C > T (R386C) | c.181C > T (R61W) | ERT |
| | | Caucasian | | | | | |
| M69 | M | Asian | −3.03 | 2.33 | c.106_111del | c.1201C > T (H401Y) | ERT |
| | | | | | (p.L36_L37del) | | |
| M71 | M | White or | −3 | 9.12 | c.1219A > C (N407H) | unknown | ERT |
| | | Caucasian | | | | | |

### 1.2.2 Genotyping

Genetic testing of the GALNS gene revealed homozygotes and compound heterozygotes. In some patients, only one mutation was detected on one allele. One patient could not be genetically tested since genomic DNA could not be obtained (Table 1). Among the detected mutations, c.448delC (H150Tfs*3), c.633+1G>C, c.946G>A (G316R), and c.1339G>C (D447H) were not listed on the ClinVar website (https://www.ncbi.nlm.nih.gov/clinvar/) and no previously published articles describing these four mutations were found. The significance of the last two variants was predicted using online bioinformatics tools, MutationTaster and PolyPhen-2. Both variants were indicated as “disease-causing” and “probably damaging with a score of 1.000” by MutationTaster and PolyPhen-2, respectively. The remaining two variants were disease-causing mutations since one causes a frameshift that reduces the length of the entire peptide to less than a third of the original protein, and another one disrupts normal splicing by destroying the GT-AG rule, the basis of splicing.

### 1.2.3.1 NT-proCNP

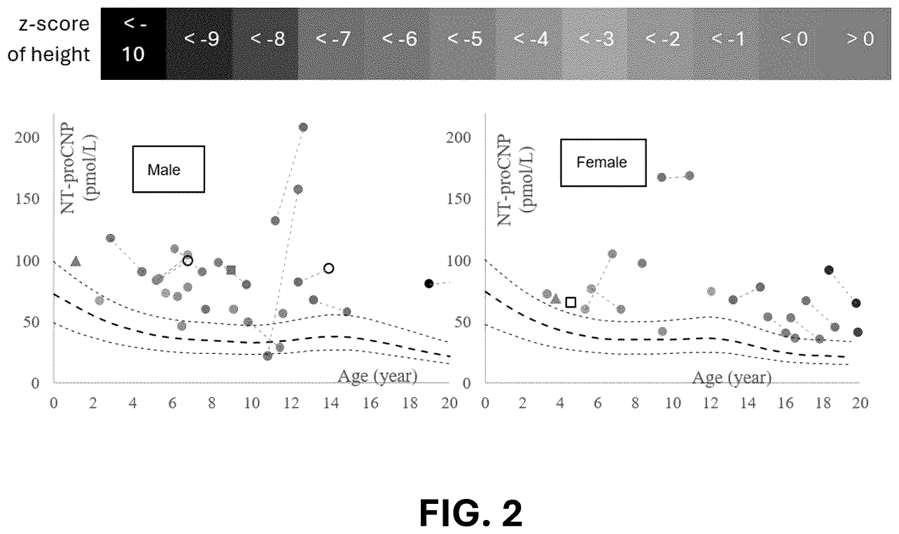

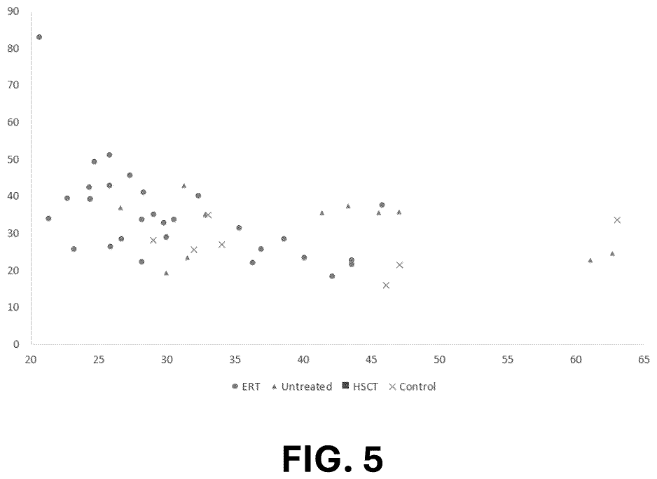

Most pediatric patients, both males and females, have elevated levels above the 95th percentile for healthy controls of both sexes, as established by Olney et al. (FIG. 2). The increase was evident just before the age of pubertal growth spurt in healthy subjects. Focusing on patients with two data points at different ages before the age of peak pubertal growth spurt (all of whom had received ERT during these two periods), boys and girls showed an increase in NT-proCNP levels. Results from adults over 20 years are summarized in FIG. 5, showing a significant difference between healthy controls and patients (p=0.019).

A higher NT-proCNP level correlated with a lower z-score for height, indicating severe growth impairment, especially between the ages of 8 and 13 years (17 subjects) (FIG. 2). Including this age group, the partial correlation coefficient between NT-proCNP level and z-score of height was calculated, with age as a controlling factor, revealing a significant negative correlation in patients older than 8 years (Table 2).

TABLE 2
The partial correlation coefficient between NT-proCNP and the
z-score of height, controlling for age, across three age groups

| | | number of | Partial correlation | |
|---|---|---|---|---|
| Age group | | subjects | coefficient | p-value |
| ≤8 | y | 20 | 0.190 | 0.4355 |
| >8, ≤13 | y | 17 | −0.568 | 0.0217 |
| >13, ≤20 | y | 15 | −0.837 | 1.865 × 10\hat{ } − 4 |

### 1.2.3.1 NT-proCNP

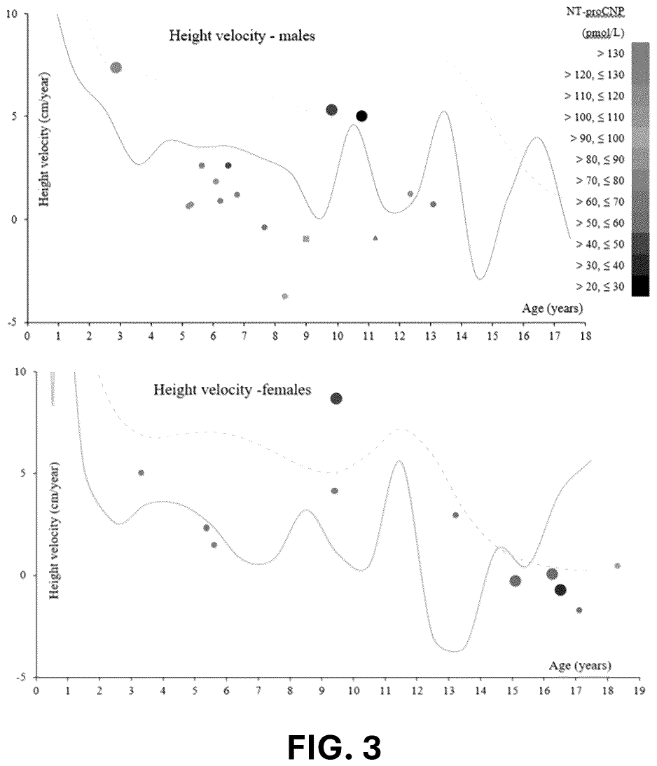

The relationship between each patient's plasma NT-proCNP level, age, and subsequent height growth rate (i.e., near-future growth rate) is shown in FIG. 3. Higher NT-proCNP levels do not lead to a higher growth rate but to a lower one, especially after the age of 8 years. This trend could not be statistically analyzed due to the limited amount of data.

### 1.2.3.2 Collagen Type I

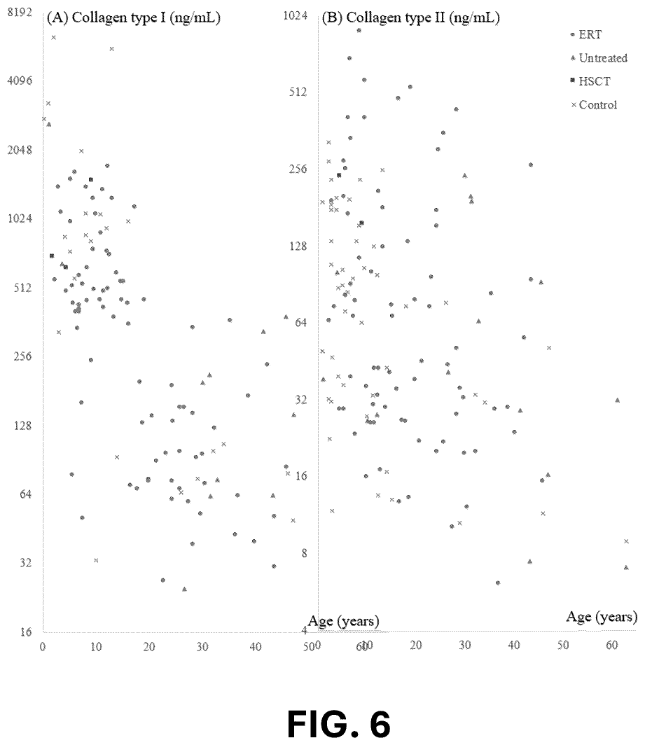

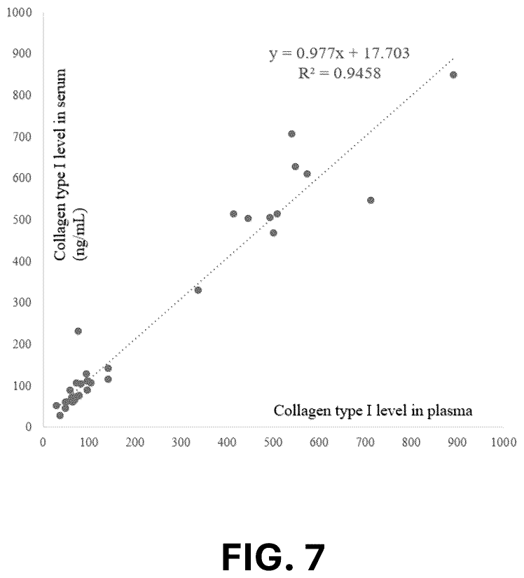

A statistically significant increase in collagen type I was observed in patients older than 20 years (Table 3, FIG. 6). The correlation between serum and plasma was straightforward, as shown in FIG. 7, indicating that the comparison between serum from controls and plasma from patients is feasible and reasonable.

TABLE 3

| Age | No. | Mean | SD | Maximum | Minimum | Mean age | p-value | Specimen |
|---|---|---|---|---|---|---|---|---|
| Control | | | | | | | | |
| ≤1 y | 1 | 2803.4 | 0 | 2803.4 | 2803.4 | 0.25 | | Serum |
| >1, ≤5 y | 4 | 2715.5 | 2400.5 | 6397.5 | 329.6 | 2.51 | | Serum |
| >5, ≤10 y | 6 | 1019.0 | 481.2 | 2039.0 | 566.0 | 7.19 | | Serum |
| >10, ≤15 y | 5 | 1572.0 | 2123.6 | 5734.8 | 32.9 | 11.98 | | Serum |
| >15, ≤20 y | 1 | 996.1 | 0 | 996.1 | 996.1 | 15.99 | | Serum |
| >20 y | 7 | 76.6 | 18.6 | 106.3 | 49.3 | 39.57 | | Plasma |
| MPS IVA | | | | | | | | |
| >1, ≤5 y | 8 | 1022.4 | 681.8 | 2654.1 | 490.7 | 3.05 | 0.31 | Plasma |
| >5, ≤10 y | 24 | 679.4 | 469.3 | 1630.2 | 50.3 | 7.38 | 0.19 | Plasma |
| >10, ≤15 y | 14 | 752.4 | 398.1 | 1722.0 | 381.2 | 12.52 | 0.48 | Plasma |
| >15, ≤20 y | 11 | 323.9 | 314.8 | 1161.6 | 67.0 | 17.70 | N/A | Plasma |
| >20 y | 41 | 125.7 | 92.1 | 383.0 | 24.5 | 33.35 | 0.0046 | Plasma |

### 1.2.3.3 Collagen Type II

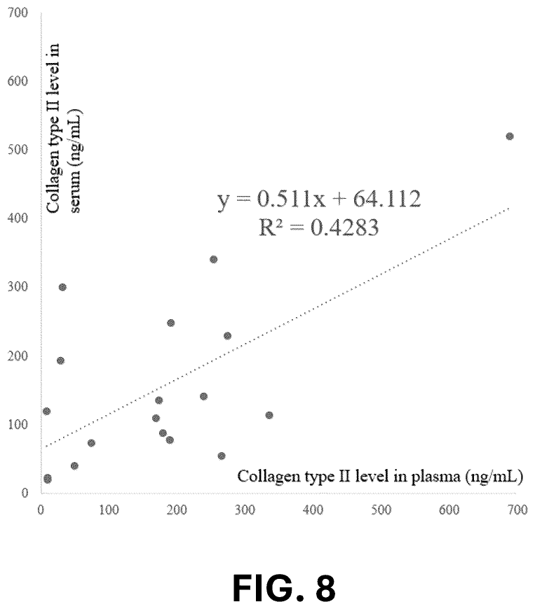

A significant increase in collagen type II was observed in patients in two age groups, between 5 and 10 years and over 20 years (Table 4, FIG. 6). However, the correlation between serum and plasma was unclear, as shown in FIG. 8, which complicated the interpretation of the results for the former age group. In addition, the partial correlation coefficient between collagen type II and NT-proCNP was calculated, controlling for age using the entire age group, since collagen type II is a marker of chondrocytes and CNP is a growth factor for chondrocytes. However, the data showed no correlation (r=0.042, p=0.69).

TABLE 4
Comparison of collagen type II levels in five age-groups

| Age | No. | Mean | SD | Maximum | Minimum | Mean age | p-value | Specimen |
|---|---|---|---|---|---|---|---|---|
| Control | | | | | | | | |
| >1, ≤5 y | 18 | 129.03 | 92.41 | 324.99 | 11.70 | 2.68 | | Serum |
| >5, ≤10 y | 12 | 113.70 | 53.62 | 231.40 | 36.66 | 6.97 | | Serum |
| >10, ≤15 y | 7 | 85.64 | 78.26 | 253.57 | 16.66 | 12.21 | | Serum |
| >15, ≤20 y | 2 | 43.85 | 30.83 | 74.68 | 13.02 | 16.50 | | Serum |
| >20 y | 7 | 35.72 | 22.85 | 76.23 | 10.51 | 39.57 | | Plasma |
| MPS IVA | | | | | | | | |
| >1, ≤5 y | 7 | 105.80 | 74.15 | 240.58 | 29.44 | 3.23 | 0.55 | Plasma |
| >5, ≤10 y | 21 | 234.54 | 233.64 | 885.55 | 15.93 | 7.30 | 0.037 | Plasma |
| >10, ≤15 y | 14 | 70.07 | 59.39 | 208.32 | 17.03 | 12.52 | 0.67 | Plasma |
| >15, ≤20 y | 11 | 131.71 | 181.03 | 534.18 | 12.77 | 17.70 | 0.21 | Plasma |
| >20 y | 40 | 90.71 | 105.27 | 438.58 | 6.10 | 33.54 | 0.0097 | Plasma |

### 1.2.3.4. Glycosaminoglycans (GAGs)

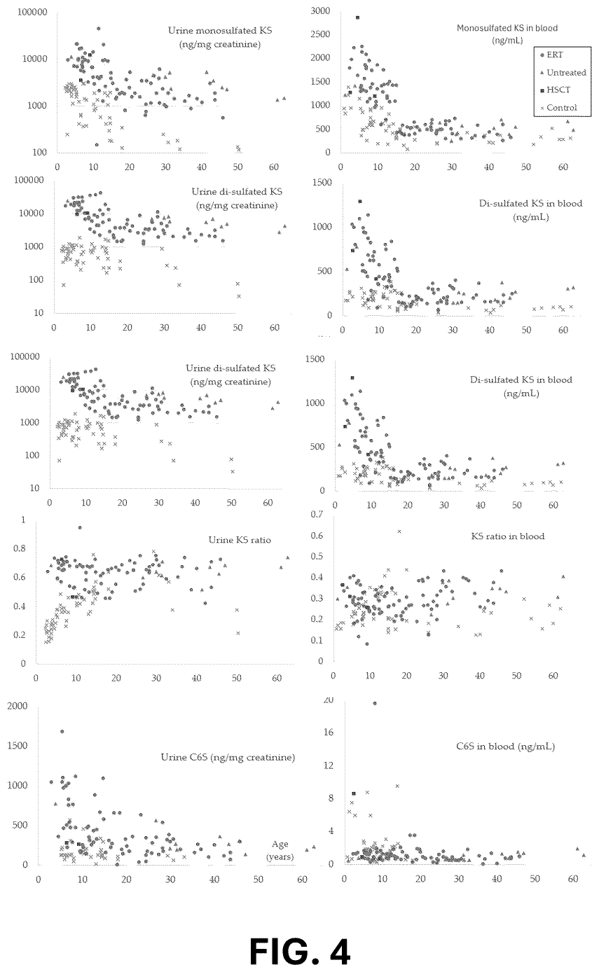

The results of mono-sulfated KS, di-sulfated KS, KS ratio (di-sulfated KS to total KS), and C6S in urine and blood from patients are summarized in Table 5 and FIG. 4. Patients had a higher sulfation level in urinary KS than age-matched controls, except for the group between 15 and 20 years. Sulfation levels of urinary KS in normal controls increased with age, while the levels in MPS IVA patients remained constant in all age groups. MPS IVA patients had higher sulfation levels in blood KS than age-matched controls up to 10 years. Sulfation levels of blood KS in normal controls increased until 15 years and then declined with age, whereas levels in MPS IVA patients remained relatively constant across all age groups. Overall, sulfation patterns and age-dependent changes differed between urinary KS and blood KS in both control and MPS IVA patient groups, indicating the difference in the origin of urinary and blood KS.

TABLE 5
Summary of GAG levels in five age-groups

| | | | | | | | Number | |
|---|---|---|---|---|---|---|---|---|
| | Number of | Mean | Mono-sulfated | Di-sulfated | KS ratio | | of | Mean |
| Age group | samples | age | KS | KS | (%) | C6S | samples | age |
| Control (urine) | | | | | | | | |
| >1, ≤5 y | 18 | 3.47 | 2128 ± 618 | 663 ± 263 | 23.4 ± 4.6 | N/A | 0 | N/A |
| >5, ≤10 y | 14 | 6.48 | 1380 ± 833 | 768 ± 439 | 36.8 ± 5.6 | 164.7 ± 129.7 | 14 | 6.48 |
| >10, ≤15 y | 15 | 12.75 | 971 ± 574 | 802 ± 413 | 46.5 ± 9.4 | 178.1 ± 104.1 | 15 | 12.75 |
| >15, ≤20 y | 3 | 17 | 215 ± 96 | 271 ± 69 | 57.2 ± 4.9 | 83.3 ± 17.2 | 3 | 17 |
| >20 y | 6 | 37.92 | 157 ± 45 | 255 ± 283 | 48.7 ± 18.6 | 88.0 ± 19.8 | 3 | 31.03 |
| MPS IVA (urine) | | | | | | | | |
| >1, ≤5 y | 3 | 3.73 | 9202 ± 1563* | 20081 ± 2603** | 68.6 ± 3.6*** | 727 ± 282 | | |
| >5, ≤10 y | 23 | 7.24 | 9904 ± 5024*** | 18225 ± 9116*** | 63.9 ± 8.3*** | 633 ± 398*** | | |
| >10, ≤15 y | 15 | 12.36 | 8143 ± 10725* | 12343 ± 11876** | 65.1 ± 11.1*** | 465 ± 247*** | | |
| >15, ≤20 y | 11 | 17.7 | 2340 ± 1178*** | 3091 ± 1509*** | 56.7 ± 8.0 | 289 ± 189** | | |
| >20 y | 39 | 33.56 | 2267 ± 1391*** | 4033 ± 2286*** | 64.4 ± 7.7 | 245 ± 131*** | | |
| Control (serum) | | | | | | | | |
| >1, ≤5 y | 4 | 2 | 1095 ± 223 | 229 ± 40 | 17.4 ± 1.3 | 5.15 ± 2.67 | 4 | 2 |
| >5, ≤10 y | 17 | 7.18 | 660 ± 220 | 197 ± 59 | 23.3 ± 3.6 | 2.34 ± 2.01 | 17 | 7.18 |
| >10, ≤15 y | 10 | 12.35 | 545 ± 235 | 248 ± 88 | 32.3 ± 5.0 | 2.81 ± 2.29 | 10 | 12.35 |
| >15, ≤20 y | 10 | 15.9 | 322 ± 123 | 118 ± 73 | 27.8 ± 14.7 | 0.49 ± 0.06 | 2 | 17 |
| >20 y | 15 | 42.2 | 314 ± 109 | 86 ± 43 | 21.4 ± 7.8 | N/A | 0 | N/A |
| MPS IVA (plasma) | | | | | | | | |
| >1, ≤5 y | 8 | 3.13 | 1805 ± 514* | 910 ± 229*** | 33.7 ± 4.1 *** | 1.97 ± 2.53 | 8 | 3.13 |
| >5, ≤10 y | 24 | 7.38 | 1522 ± 369*** | 597 ± 258*** | 27.2 ± 7.7* | 1.97 ± 3.88 | 22 | 7.33 |
| >10, ≤15 y | 14 | 12.52 | 1257 ± 306*** | 509 ± 181*** | 28.4 ± 4.3 | 1.12 ± 0.41 | 14 | 12.52 |
| >15, ≤20 y | 11 | 17.7 | 482 ± 90** | 166 ± 45 | 25.5 ± 4.6 | 1.65 ± 1.04 | 11 | 17.7 |
| >20 y | 41 | 33.35 | 493 ± 131*** | 234 ± 79*** | 32.1 ± 6.9 *** | 0.85 ± 0.43 | 40 | 33.54 |

For C6S, the number of available control samples was less than for KS, so the “number of samples” and “mean age” differed from those of KS.
Items that showed a significant increase in patients are marked with an asterisk (*p < 0.05, **p < 0.01, ***p < 0.001). No items showed a significant decrease in MPS IVA patients.

Partial correlation coefficients, controlling for age, between the z-score of height and each significant GAG (mono- and di-sulfated KS and urinary C6S) were calculated and summarized in Table 6. Both urinary KS showed a moderate negative correlation with z-score of height with statistical significance.

TABLE 6
Partial correlation coefficient (r) between each GAG and the z-
score of height of patients under 20 years, controlling for age

| | Number of subjects to | | |
|---|---|---|---|
| | calculate r value | r | p |
| Urine di-sulfated KS | 49 | −0.484 | 0.00049 |
| Urine mono-sulfated KS | 49 | −0.42 | 0.00293 |
| Plasma di-sulfated KS | 52 | −0.048 | 0.740 |
| Plasma mono-sulfated KS | 52 | −0.186 | 0.192 |
| Urine C6S | 49 | −0.282 | 0.0517 |

### 1.2.3.5. Effect of ERT on Each Biomarker in Adult Patients Aged 25-50 Years

Two biomarkers, urinary mono-sulfated KS and di-sulfated KS, exhibited a significant decrease in patients treated with ERT, whereas other biomarkers showed no significant differences (Table 7).

TABLE 7
Differences in each biomarker between adult patients aged 25-50 years with and without ERT

| | | | Mean | number of | | | |
|---|---|---|---|---|---|---|---|
| | Biomarker | group | age | subjects | Mean | SD | p-value |
| Urine | C6S | No ERT | 37.04 | 8 | 285 | 123 | 0.38 |
| | | ERT | 33.49 | 21 | 237 | 109 | |
| | Mono-sulfated KS | No ERT | 37.04 | 8 | 3648 | 1146 | 0.0073 |
| | | ERT | 33.49 | 21 | 2005 | 1320 | — |
| | Di-sulfated KS | No ERT | 37.04 | 8 | 6102 | 1247 | 0.0012 |
| | | ERT | 33.49 | 21 | 3540 | 2254 | |
| | KS ratio | No ERT | 37.04 | 8 | 0.631 | 0.057 | 0.74 |
| | | ERT | 33.49 | 21 | 0.641 | 0.088 | |
| Plasma | C6S | No ERT | 36.57 | 9 | 0.913 | 0.400 | 0.57 |
| | | ERT | 33.49 | 21 | 0.814 | 0.428 | |
| | Mono-sulfated KS | No ERT | 36.57 | 9 | 492 | 134 | 0.58 |
| | | ERT | 33.15 | 22 | 460 | 135 | |
| | Di-sulfated KS | No ERT | 36.57 | 9 | 226 | 68.4 | 0.89 |
| | | ERT | 33.15 | 22 | 221 | 84.0 | |
| | KS ratio | No ERT | 36.57 | 9 | 0.317 | 0.053 | 0.79 |
| | | ERT | 33.15 | 22 | 0.324 | 0.076 | |
| | NT-proCNP | No ERT | 36.57 | 9 | 32.5 | 6.54 | 0.77 |
| | | ERT | 33.15 | 22 | 31.6 | 8.63 | |
| | Collagen type I | No ERT | 36.57 | 9 | 165.4 | 118.8 | 0.32 |
| | | ERT | 33.15 | 22 | 117.2 | 90.8 | |
| | Collagen type II | No ERT | 36.57 | 9 | 98.7 | 84.3 | 0.63 |
| | | ERT | 33.49 | 21 | 79.5 | 116.6 | |

### 1.2.3.6. Transitions in Skeletal Growth and Biomarkers in Pediatric Patients Younger than 12 Years with More than Two Hospital Visits (Table 8)

Patients who met the criteria described in the Materials and Methods section received ERT or HSCT. Height was categorized into percentiles based on the growth chart constructed from accumulated data of Morquio A patients without ERT or HSCT. Red-colored cells indicate improvement in the percentile category. Blue-colored cells indicate a worsening of the percentile category. Pink-colored cells indicate an increase of more than 6% of the biomarker value. Light blue-colored cells indicate a decrease of more than 7%. Gray-colored cells indicate almost no change in the biomarker over the two time points.

Two of the patients who routinely received ERT worsened in the height percentile category. Some showed improvement, and others were not affected in their height (Table 8). Among patients with ERT, some had decreased NT-proCNP levels in plasma, some had increased, and some had the same level. Some patients with ERT had increased blood mono-sulfated KS levels, and others had decreased. Most patients with ERT had decreased mono-sulfated KS levels in urine, while others had increased levels. Among patients with ERT, some had increased blood di-sulfated KS levels, and others had decreased. Some patients with ERT had increased urinary di-sulfated KS levels, while others had decreased levels. No correlation was found in two-point changes (decrease, increase, or no change) between NT-proCNP and urinary KS or blood KS.

Sulfation levels (KS ratio) in the urine of all patients were higher than in the blood. Most patients with ERT had increased sulfation levels in urine, while others had the same level. Among patients with ERT, some had increased blood sulfation levels, some had decreased, and some had the same level.

TABLE 8

| | | | | | NT-proCNP | Mono-sulfated KS | | Di-sulfated KS | | | | |
|---|---|---|---|---|---|---|---|---|---|---|---|---|
| | | Height | Age | Percentile | plasma | Plasma | Urine | Plasma | Urine | KS ratio | | |
| ID | Sex | (cm) | (year) | of height | (pmol/L) | (ng/mL) | (ng/mg cre) | (ng/mL) | (ng/mg | Plasma | Urine | Treatmen |
| M52 | F | | 1.84 | | | | | | | | | HSCT |
| | | 83 | 2.16 | 25th-50th* | no data | no data | no data | no data | no data | no data | no data | |
| | | No record | 2.5 | | no data | 1277.0 | no data | 739.0 | no data | 0.3668 | no data | |
| | | 91.5 | 3.14 | 75th* | | | | | | | | |
| | | No record | 4.6 | | 64.94 | 2864.2 | no data | 1297.2 | no data | 0.3117 | no data | |
| | | 104 | 6.21 | 75th-90th* | no data | no data | 3564 | no data | 9502 | no data | 0.7272 | |
| M51 | M | 91.5 | 2.89 | 50th-75th* | 117.43 | 1977.9 | 9658 | 1005.9 | 17357 | 0.3371 | 0.6425 | ERT |
| | | 103.3 | 4.5 | 75th-90th* | 90.39 | 1643.9 | 7102 | 1098.5 | 19299 | 0.4006 | 0.7310 | |
| M16 | M | 85.6 | 5.21 | 3rd-10th | 83.29 | 1305.0 | 20783 | 509.0 | 30026 | 0.2806 | 0.5910 | ERT |
| | | 86.2 | 6.23 | 3rd-10th | no data | no data | no data | no data | no data | no data | no data | |
| | | No record | 6.77 | No record | 99.67 | 1818.3 | 7679 | 690.5 | 17611 | 0.2752 | 0.6964 | |
| M15 | M | 99.5 | 5.32 | 50th-75th | 85.15 | 1378.1 | 21141 | 578.0 | 30952 | 0.2955 | 0.5942 | ERT |
| | | 100.5 | 6.81 | 50th** | 103.81 | 2078.6 | 8783 | 878.7 | 21695 | 0.2971 | 0.7118 | |
| M55 | F | 96.7 | 5.39 | 50th-75th | 60.13 | 1426.9 | 6701 | 996.6 | 16964 | 0.4112 | 0.7168 | ERT |
| | | 99.9 | 6.8 | 50th-75th | 104.42 | 1310.0 | no data | 182.2 | no data | 0.1221 | no data | |
| M50 | F | 97 | 5.65 | 50th-75th | 76.66 | 2134.8 | 10127 | 949.8 | 19754 | 0.3079 | 0.6611 | ERT |
| | | 99.3 | 7.26 | 50th-75th | 59.80 | 1079.4 | 5293 | 627.5 | 15186 | 0.3676 | 0.7415 | |
| M21 | M | 95 | 6.12 | 25th-50th | 109.48 | 1328.6 | 16376 | 496.0 | 21635 | 0.2718 | 0.5692 | ERT |
| | | 97.6 | 7.55 | 25th-50th | 90.06 | 1945.9 | 13424 | 834.9 | 32474 | 0.3003 | 0.7075 | |
| M46 | F | 96.6 | 6.51 | 50th | no data | no data | no data | no data | no data | no data | no data | ERT |
| | | No record | 6.92 | No record | no data | no data | 6476 | no data | 12382 | no data | 0.6566 | |
| | | 100.6 | 8.4 | 50th | 97.35 | 1470.7 | 3246 | 723.3 | 6846 | 0.3297 | 0.6784 | |
| M08 | M | 96.4 | 7.86 | 25th | no data | no data | no data | no data | no data | no data | no data | ERT |
| | | No record | 8 | | no data | 1377.9 | no data | 674.3 | no data | 0.3286 | no data | |
| | | | 8.07 | | | | | | | | | HSCT |
| | | 99 | 9.03 | 25th | 91.53 | 1196.0 | 12063 | 418.0 | 10503 | 0.2588 | 0.4654 | |
| | | 97.6 | 10.42 | 10th-25th** | no data | no data | no data | no data | no data | no data | no data | |
| M22 | M | 100 | 8.34 | 25th-50th | 98.16 | 1746.7 | 8848 | 626.6 | 10450 | 0.2640 | 0.5415 | ERT |
| | | 94.6 | 9.78 | 10th-25th** | 79.68 | 1859.7 | 13776 | 579.1 | 35514 | 0.2375 | 0.7205 | |
| M19 | F | 98.5 | 9.44 | 25th-50th* | 167.19 | 1081.4 | 6616 | 363.0 | 7330 | 0.2513 | 0.5256 | ERT |
| | | 104.7 | 10.96 | 50th* | 168.59 | 1415.3 | 4146 | 411.4 | 9157 | 0.2252 | 0.6883 | |
| M20 | F | 111.1 | 9.49 | 75th-90th | 41.26 | 1005.8 | 6480 | 353.7 | 7416 | 0.2602 | 0.5337 | ERT |
| | | 116 | 10.06 | 75th-90th | no data | no data | 2903 | no data | 6026 | no data | 0.6749 | |
| M31 | M | 129.5 | 9.86 | 90th | 49.42 | 1359.5 | 4043 | 445.9 | 3538 | 0.2470 | 0.4667 | ERT |
| | | 137.8 | 11.44 | 90th-97th* | 28.52 | 1760.7 | 2116 | 808.5 | 4436 | 0.3147 | 0.6770 | |

*indicates improvement in the percentile category.
**indicates a worsening of the percentile category.
(bold) indicates an increase of more than 6% of the biomarker value.
(italics) colored cells indicate a decrease of more than 7%.
Gray-colored cells indicate almost no change in the biomarker over the two-time points.

### 1.2.3.7. Correlation Between NT-proCNP and Other Biomarkers

The correlation between NT-proCNP and other biomarkers was examined in the three pediatric age groups and summarized in Table 9. Only plasma and urinary C6S showed correlations with statistical significance (p<0.05) over 13 years of age, suggesting that NT-proCNP can be an independent biomarker from KS.

TABLE 9
Correlation between NT-proCNP and other
biomarkers in pediatric age groups

| | | | number of |
|---|---|---|---|
| | r | p | patients |
| Plasma C6S | | | |

| | | | | |
|---|---|---|---|---|
| ≤8 | y | −0.043 | 0.8543 | 21 |
| >8, ≤13 | y | 0.116 | 0.6682 | 16 |
| >13, ≤20 | y | 0.503 | 0.0472 | 16 |

Urinary C6S

| | | | | |
|---|---|---|---|---|
| ≤8 | y | 0.365 | 0.1501 | 17 |
| >8, ≤13 | y | 0.425 | 0.1011 | 16 |
| >13, ≤20 | y | 0.634 | 0.0084 | 16 |

Plasma mono-sulfated KS

| | | | | |
|---|---|---|---|---|
| ≤8 | y | −0.056 | 0.8048 | 22 |
| >8, ≤13 | y | 0.176 | 0.4986 | 17 |
| >13, ≤20 | y | 0.387 | 0.1382 | 16 |

Urinary mono-sulfated KS

| | | | | |
|---|---|---|---|---|
| ≤8 | y | 0.097 | 0.7113 | 17 |
| >8, ≤13 | y | 0.364 | 0.1658 | 16 |
| >13, ≤20 | y | 0.429 | 0.0974 | 16 |

Plasma di-sulfated KS

| | | | | |
|---|---|---|---|---|
| ≤8 | y | −0.075 | 0.7406 | 22 |
| >8, ≤13 | y | −0.080 | 0.7615 | 17 |
| >13, ≤20 | y | 0.363 | 0.1672 | 16 |

Urinary di-sulfated KS

| | | | | |
|---|---|---|---|---|
| ≤8 | y | −0.085 | 0.7465 | 17 |
| >8, ≤13 | y | 0.458 | 0.0747 | 16 |
| >13, ≤20 | y | 0.483 | 0.0580 | 16 |

Plasma KS ratio

| | | | | |
|---|---|---|---|---|
| ≤8 | y | −0.095 | 0.6736 | 22 |
| >8, ≤13 | y | −0.179 | 0.4913 | 17 |
| >13, ≤20 | y | 0.007 | 0.9803 | 16 |

Urinary KS ratio

| | | | | |
|---|---|---|---|---|
| ≤8 | y | −0.323 | 0.2054 | 17 |
| >8, ≤13 | y | −0.156 | 0.5633 | 16 |
| >13, ≤20 | y | −0.147 | 0.5861 | 16 |

TABLE 10
PCR

| PCR 1: | PCR 2: | | | | | PCR 3: | | |
|---|---|---|---|---|---|---|---|---|
| 93 °C | 3 min | 1 cycle | 93 °C | 3 min | 1 cycle | 98 °C | 5 min | 1 cycle |
| 93 °C | 30 sec | 40 cycles | 93 °C | 30 sec | 35 cycles | 98 °C | 30 sec | 35 cycles |
| 70 °C | 30 sec | | 67 °C | 3 min | | 63 °C | 30 sec | |
| 72 °C | 10 min | 1 cycle | 72 °C | 10 min | 1 cycle | 72 °C | 5 min | 1 cycle |

| SEQ | | | | Amplified | Nucleotide length of | PCR |
|---|---|---|---|---|---|---|
| ID | | Primer sequence | | exons | amplified DNA | condition |
| 1 | 5′ | CTGGCTCAGGCCCCGCCCCACT | 3′ | Exon 1 | 393 base pairs | PCR 1 |
| 2 | 5′ | CTTCCCCCACCTCGCTCCTCCCTCCAT | 3′ | | | |
| 3 | 5′ | CTGGCTTCCACGGTCCCCGACAC | 3′ | Exon 2, 3, | 2050 base pairs | PCR 2 |
| 4 | 5 | CCCAAGACACCCTCCTCATTTGGAAACT | 3 | 4 | | |
| 5 | 5′ | CTCAGTGCTGGAGGGTGCTCGTCTTA | 3′ | Exon 5, 6, | 2822 base pairs | PCR 2 |
| 6 | 5′ | AGGCTCGGTGACATCTGCTCCTCC | 3′ | 7, 8 | | |
| 7 | 5′ | CCCTTTGTCCCTATGACCAGTCTCAGT | 3 | Exon 9 | 261 base pairs | PCR 3 |
| 8 | 5′ | GGGATGGCTGCAGGCCTGGACCT | 3′ | | | |
| 9 | 5′ | TGTGAGCATGTATGCATATCTGTAGACCCA | 3′ | Exon 10 | 490 base pairs | PCR 3 |
| 10 | 5′ | AAGCCAGGGCCTCGCTGTGCTT | 3′ | | | |
| 11 | 5′ | TGATGACAGACGCAGCCCAGAGCC | 3′ | Exon 11, 12 | 2511 base pairs | PCR 2 |
| 12 | 5′ | GCACGTGTGGGTATGAATAGCAACAGCA | 3′ | | | |
| 13 | 5′ | TGCTCACTGTGGTTCTCAGCCCGTT | 3′ | | 3926 base pairs | PCR 2 |
| 14 | 5′ | GTCTGCAGGTGCTGTCTGTCTGGCTT | 3′ | Exon 13, 14 | | |

### 1.3 Conclusions

In this study, several biomarkers were measured in MPS IVA patients enrolled as part of the Morquio A natural history program to evaluate the significance of each biomarker in relation to growth failure. Most patients received ERT, and a few received HSCT. Some pediatric patients had not yet started ERT due to recent diagnosis, while some adult patients had never received ERT despite its approval. Judging from their heights, ERT did not show apparent effects for skeletal dysplasia, including bone growth (FIG. 1, Table 8), as indicated by previous reports. Four novel mutations were identified through genetic testing; one was a deletion resulting in a frameshift, another was a mutation at a splice donor site, and the remaining two were missense mutations predicted to be disease-causing by bioinformatics tools. In some patients, only one mutation was found on one allele. Sequencing was limited to the exons and exon-intron boundaries of the GALNS gene, without searching for large deletions, thereby limiting genetic testing.

CNP is significantly involved in both vascular homeostasis and bone growth, which is mainly controlled by CNP secreted from vascular endothelial cells and chondrocytes in growth plates, respectively. It plays a key role in endochondral ossification, crucial for forming and growing long bones. CNP primarily affects bone growth by signaling through the chondrocyte receptor NPR-B, encoded by the NPR2 gene, which induces cell division and differentiation.

Recent research indicated that CNP and NT-proCNP levels are significantly elevated in several skeletal dysplasias caused by mutations in the FGFR3 or NPR2 gene. These genes are closely related to each other and to extracellular CNP, playing a crucial role in cell proliferation and differentiation. Substantial progress has been made in recent years in the study of CNP and their role in skeletal disorders, particularly achondroplasia, which is caused by a gain-of-function mutation in FGFR3 that results in constitutive activation of FGFR3 and the downstream MEK/ERK MAPK pathway. The MEK/ERK MAPK pathway regulates various cellular processes, including cell growth, proliferation, and differentiation. Elevated levels of CNP and NT-proCNP in achondroplasia are thought to be a natural response of the human body to this overactivation of the MEK/ERK MAPK pathway in chondrocytes, which functionally inhibits the effect of CNP on chondrocytes via NPR-B encoded by NPR2. This disclosure identifies the elevation of NT-proCNP in MPS IVA patients. The gene responsible for MPS IVA is GALNS, which is not closely related to FGFR3 or NPR2. Therefore, this is the first report identifying the elevation of NT-proCNP in patients with a disease unrelated to mutations in the FGFR3 or NPR2 genes.

Recent research has not only identified NT-proCNP as a potential biomarker but also highlighted the therapeutic potential of CNP. The development of drugs such as Vosoritide, a CNP analog approved to induce bone growth in achondroplasia, underscores the potential of CNP as a therapeutic agent against the growth impairment of MPS. This potential has already been demonstrated in MPS IVA and VII mouse models.

NT-proCNP is a peptide fragment derived from the precursor protein of CNP. The NPPC gene expresses a precursor protein known as preproCNP. PreproCNP undergoes proteolytic cleavage to remove the signal peptide and to form proCNP. Furin, one of the proprotein convertases, cleaves NT-proCNP from proCNP in the trans-Golgi network to produce the active CNP. After the cleavage of proCNP, active CNP is secreted into the extracellular space via exocytosis. For NT-proCNP, no clear mechanism of secretion by cells has been found; however, these processes should occur primarily in neurons, glial cells, vascular endothelial cells, and chondrocytes in cartilage. The half-life of CNP in the blood is short, as NPRs take up CNP, and such a short half-life helps to adapt to frequent changes in circulatory status. In the case of NT-proCNP, there is no clear pathway to remove it. As a result, the normal concentration ranges of these two molecules in healthy children are significantly different, making NT-proCNP easier to detect and quantify than CNP. Considering that bone growth is a slower and more gradual phenomenon than the adaptation of the circulatory system, NT-proCNP is expected to be a better biomarker than CNP for correlating human skeletal symptoms in MPS. This leads to the selection of NT-proCNP as a potential surrogate biomarker for future studies.

NT-proCNP was significantly elevated in the plasma of MPS IVA patients in all comparable age groups. In addition, the concentrations in patients negatively correlated with height z-score, the most reliable indicator of skeletal growth failure. The source of this elevated NT-proCNP (presumably, CNP as well) remains unknown. The primary sources of these peptides are neurons, glial cells, vascular endothelial cells, and chondrocytes in cartilage, but the exact percentage of production at each site under normal conditions remains uncertain. The CNP production by growth plate chondrocytes is the most critical factor for bone growth, as evidenced by the growth impairment observed in mice where the Nppc gene has been selectively knocked out in chondrocytes. Even when plasma CNP levels were significantly reduced by knocking out the Nppc gene selectively in mouse vascular endothelial cells, Moyes et al. did not observe any growth failure.

Potential hypotheses contributing to plasma NT-proCNP elevation in MPS IVA patients include:
1—The expression of the NPPC gene is upregulated in MPS IVA patients to compensate for growth impairment or tissue resistance to CNP, as seen in other skeletal dysplasias. A feedback mechanism in patients with growth failure contributes to the higher expression of CNP, possibly involving vascular endothelial cells as a source of excessive CNP and NT-proCNP secretion.
2—CNP and NT-proCNP are leaking from damaged chondrocytes in MPS IVA patients, with secretion or leakage of NT-proCNP from damaged chondrocytes in the growth plates entering the bloodstream.
3—NT-proCNP clearance is delayed due to a systemic disorder of MPS IVA.

A combination of factors may account for the elevation of NT-proCNP. With age, the contributing factor could vary. Further research is needed to investigate regulation of CNP expression and clarify this paradoxical phenomenon.

Focusing on patients who had two data points at different ages prior to peak pubertal growth spurt (all of whom had received ERT during these two periods), NT-proCNP levels increased in some boys and girls (FIG. 2) while urinary mono-sulfated KS was reduced in most patients in Table 8. This finding indicates that plasma NT-proCNP is not a pharmacodynamic biomarker like urinary KS prior to the onset of pubertal growth spurt. Therefore, it is hypothesized that reduced NT-proCNP can be more directly linked to improving bone growth than reducing total urinary KS.

Collagen Type I is the most abundant collagen in the human body. It is a fibrous protein that forms a significant part of the extracellular matrix in various tissues, including skin, tendons, ligaments, and bone. It provides a scaffold for bone mineralization and forms a fibrous network that serves as the structural framework for bone tissue. This network supports the deposition of hydroxyapatite crystals, primarily composed of calcium and phosphate. The interaction between collagen fibers and these crystals gives bones strength and rigidity. Procollagen Type I N-Terminal Propeptide (P1NP) and C-Terminal Telopeptide of Type I Collagen (CTX-1) are two major peptides well studied in several diseases related to collagen type I metabolism. Elevated levels of these peptides are associated with diseases or conditions related to increased bone turnover, fibrotic processes, or cancer.

Several papers reported that mRNA encoding collagen type I increased in chondrocytes from human MPS IVA patients. Comprehensive proteomic analysis of mouse femurs also showed that collagen alpha-1 and 2 increased in MPS IVA. Therefore, higher concentration levels than the control group were predicted. However, only adult patients showed such differences. Other age groups showed lower concentrations in patients. Since human collagen type I has a high molecular weight and is surrounded by hydroxyapatite in bone, it could be difficult for collagen type I to escape from bone and cartilage and enter the bloodstream compared to NT-proCNP, thereby preventing higher concentrations in the blood of patients. Measurement of smaller peptides related to collagen type I may be needed for future studies.

Collagen Type II is a specific type of collagen found predominantly in cartilage and is a marker for chondrocytes. It is also present in the vitreous humor of the eye and in the nucleus pulposus of intervertebral discs. Like other collagens, Type II collagen is a fibrous protein that forms a triple-helical structure, providing tensile strength and structural integrity to tissues. Its primary role in forming and maintaining cartilage makes it crucial for joint health. Blood procollagen II C-terminal propeptide (CPII) and/or urinary C-terminal telopeptide of type II collagen (CTX-II) are currently the main biomarkers of type II collagen metabolism in osteoarthritis, joint injuries, and Kashin-Beck disease. CPII reflects the synthesis of type II collagen during cartilage repair and remodeling, while CTX-II is a marker of type II collagen breakdown, reflecting cartilage catabolism.

Previous studies reported decreased expression of mRNA encoding collagen type II in chondrocytes from human MPS IVA patients, in contrast to collagen type I. The same phenomenon was observed in mouse models of MPS type I and type VII. These findings suggest that the decrease in collagen type II may be a potential biomarker for MPS types I, IVA, and VII. In contrast, proteomic analysis of mice revealed a statistically significant increase of collagen alpha-1(II) chain in the MPS IVA model. Results showed a statistically significant increase in patients aged 5 to 10 years and over 20 years, supporting the results of the latter proteomic analysis. However, due to the poor correlation between the measured concentration in serum and plasma, as shown in FIG. 8, it is too early to draw conclusions for patients aged 5 to 10 years.

The correlation coefficient between collagen type II and NT-proCNP, controlling for age, was calculated to evaluate the effect of excessive CNP expression on chondrocytes in patients. NT-proCNP concentration would indicate the total expression level of CNP in a subject's body. Collagen type II is a marker of chondrocytes. Therefore, elevated NT-proCNP levels could correlate positively with collagen type II levels. However, no correlation was found, suggesting that the increased CNP secretion in patients is insufficient to compensate for the damage to chondrocytes in growth plates.

GAG levels are well-established biomarkers for MPS, and the difference between plasma and serum is negligible, making comparison between controls and patients reasonable. The results were consistent with previous reports. However, no prior report was found that quantitatively correlates GAG levels with the severity of skeletal symptoms. Therefore, each GAG was correlated with the z-score of height, one of the quantitative indicators of growth failure. Since each GAG level except for plasma C6S tended to decrease with age up to 20 years (FIG. 4), partial correlation coefficients controlling for age were calculated, then a moderate negative correlation was found in urinary mono- and di-sulfated KS (Table 6). These two biomarkers also showed statistical differences between adult patients with and without ERT (Table 7), making urinary KS a more important prognostic biomarker than C6S in MPS IVA.

The KS assay was established by LC-MS/MS, measuring both mono- and di-sulfated KS in age-matched controls and MPS IVA patients. Previous studies identified differences in age and sulfation between urine and blood KS in MPS IVA and healthy controls, suggesting the importance of measuring both types of KS in MPS IVA. Urinary KS is considered a pharmacodynamic biomarker that does not accurately reflect the therapeutic effect on skeletal symptoms in patients receiving ERT. In this study, the transition of these two types of KS during ERT or after HSCT was observed in the most critical age group for treatment of skeletal growth, namely, those under 10 years old. Interestingly, a decrease in urinary di-sulfated KS was observed in some patients, whereas most patients showed a decrease in mono-sulfated KS (Table 8). Based on this result, urinary di-sulfated KS may not be a pharmacodynamic biomarker like mono-sulfated KS. Furthermore, blood di-sulfated KS showed more pronounced differences from controls in most age groups (FIG. 4). Further investigations are needed to clarify the significance of di-sulfated KS.

### 1.4 Methods

Subjects: MPS IVA patients receiving care at Nemours Children's Health in Delaware and participating in the natural history program (Non-invasive Functional Assessment and Pathogenesis of Morquio A, clinicaltrial.gov: NCT05284006, NIH funding number: 1R01HD102545-01A1) were enrolled. The diagnosis of MPS IVA was confirmed by deficient enzyme activity of less than 5% of normal activity measured in plasma, leukocytes, or fibroblasts before enrollment in this clinical trial. B. A total of 60 participants were part of the research study. The z-score of each patient's height at the time of sample collection was calculated based on the CDC Growth Chart. It was also categorized into percentiles based on the growth chart constructed from the accumulated data of Morquio A patients without ERT or HSCT (hematopoietic stem cell transplantation). If a patient had two or more height measurements taken at different times, the height growth rate (annual height velocity) was calculated using two data points.

Genotyping: Genomic DNA was extracted from the white blood cells and/or skin fibroblasts. The exons of the GALNS gene were amplified by polymerase chain reaction (PCR) and sequenced using the Sanger method to identify pathogenic mutations. PCR conditions and primer pairs used are summarized in Table S3. Mutations were annotated based on the reference sequence NM_000512.5.

Control Specimens: Serum or urine samples from individuals without skeletal dysplasia at Shimane University Hospital (Japan) were provided as control samples for this research. The age ranged from infancy to adulthood for serum and from childhood to adulthood for urine. Additionally, blood samples were collected from healthy adults working in the laboratory, and both plasma and serum were tested to confirm the correlation between serum and plasma for collagen type I and II levels.

ELISA-based method for the measurement of collagen type I, II, and NT-proCNP: The Human Collagen Type I ELISA Kit, the Human Collagen Type II ELISA Kit, and the NT-proCNP ELISA kit were utilized to measure the concentration of each biomarker in patient plasma and serum, control serum from children, and control plasma and serum from adults according to the manufacturer's instructions. Each kit used a quantitative sandwich enzyme immunoassay technique. The absorbance of each well was measured using FLUOstar Omega.

Glycosaminoglycans (GAGs) Analysis by LC-MS/MS: Mono-sulfated KS, di-sulfated KS, and C6S in plasma and urine samples from patients were measured according to an established method. Serum, plasma, urine samples, or standards were mixed with Tris-hydrochloric acid buffer in wells of filter plates equipped with ultrafiltration membrane filters. Recombinant chondroitinase ABC and keratanase II, and internal standard solution, were added to each well, followed by additional buffer. After overnight incubation at 37 °C, the filter plate was centrifuged. The filtered samples were injected into a liquid chromatography system with a triple quad mass spectrometer. Levels of GAGs in urine samples were normalized to creatinine, which was measured with a Creatinine (urinary) Colorimetric Assay Kit. The serum and urine of control subjects were also measured in the same way.

Effect of ERT on Each Biomarker: The difference in each biomarker between patients treated with ERT and patients who had not been treated with ERT was compared. Since most patients in the latter group were older adults, only this age range was used.

Transitions in skeletal growth and biomarkers in pediatric patients younger than 12 years with more than two hospital visits: To explore a correlation between significant biomarkers, skeletal growth, and treatment (ERT or HSCT) in growing pediatric patients, data from patients who provided their first specimen before the age of 10 years were summarized.

Statistical Analysis: Welch's t-test was used to determine statistical differences between subjects and controls for all biomarkers. Correlation coefficients or partial correlation coefficients, controlling for age, were calculated to reveal the relationship between each factor.

While preferred embodiments of the present disclosure have been shown and described herein, it will be obvious to those skilled in the art that such embodiments are provided by way of example only. Numerous variations, changes, and substitutions will now occur to those skilled in the art without departing from the instant disclosure. It should be understood that various alternatives to the embodiments described herein may be employed in practicing the disclosure. It is intended that the following claims define the scope of the embodiments disclosed herein, and that methods and structures within the scope of these claims and their equivalents be covered thereby.

1. A method of identifying skeletal growth failure in an individual having Mucopolysaccharidoses (MPS) Type IVA, the method comprising:
obtaining a biological sample from the individual;
detecting the presence of N-terminal pro-C-type natriuretic peptide (NT-proCNP) polypeptide and generating a measured concentration of NT-proCNP polypeptide in the biological sample; and
identifying the individual as having skeletal growth failure or an increased risk of skeletal growth failure if the measured concentration of NT-proCNP is greater than an age- and sex-matched reference concentration, the age- and sex-matched reference concentration comprising the mean concentration of NT-proCNP polypeptide in age- and sex-matched healthy individuals.

2. The method of claim 1, wherein the age of the individual is 8 years or greater.

3. (canceled)

4. The method of claim 1, wherein the presence of N-terminal pro-C-type natriuretic peptide (NT-proCNP) polypeptide is detected by an affinity-based detection assay.

5. The method of claim 4, wherein the affinity-based detection assay is an ELISA.

6. The method of claim 1, wherein the individual is identified as having skeletal growth failure or increased risk of skeletal growth failure if the measured concentration the measured concentration of NT-proCNP is equal to or greater than 2.0 standard deviations above the reference concentration.

7. A method of treating skeletal growth failure in an individual having MPS IVA, the method comprising:
measuring a concentration of N-terminal pro-C-type natriuretic peptide (NT-proCNP) in a blood plasma sample obtained from the individual;
determining that the concentration of NT-proCNP is equal to or greater than 1.0 standard deviations above an age- and sex-matched reference concentration; and
administering to the patient an effective amount of a C-type natriuretic peptide (CNP) analog, the CNP analogue comprising vosoritide or navepegritide, and the CNP analog being administered based on the determination that the measured NT-proCNP concentration is equal to or greater than the 1.0 standard deviation above the age- and sex-matched reference concentration.

8. The method of claim 7, wherein the measured concentration of NT-proCNP is equal to or greater than 2.0 standard deviations above an age- and sex-matched reference concentration.

9. The method of claim 7, wherein the concentration of N-terminal pro-C-type natriuretic peptide (NT-proCNP) polypeptide is measured by an affinity-based detection assay.

10. The method of claim 9, wherein the affinity-based detection assay is an ELISA.

11. The method of claim 7, wherein the individual has received and/or is concurrently receiving elosulfase alfa.

12. The method of claim 7, wherein the CNP analogue reduces the concentration of NT-proCNP in the individual.

13. A clinical decision-support (CDS) system for identifying skeletal growth failure in Mucopolysaccharidosis Type IVA (MPS IVA), the system comprising:
(a) a network communication interface configured to obtain laboratory data packets from a clinical data repository for a patient;
(b) a processor; and
(c) a non-transitory computer-readable memory storing instructions that, when executed by the processor, cause the system to:
(i) extract a first digital payload and a second digital payload from the clinical data repository, the first digital payload quantifying a plasma N-terminal pro-C-type natriuretic peptide (NT-proCNP) concentration for the patient, and the second digital payload identifying a chronological age of the patient;
(ii) query an age-stratified reference data structure using the second digital payload to retrieve an age- and sex-matched reference concentration;
(iii) execute a clinical logic gate detects a skeletal growth failure state when the plasma NT-proCNP concentration is greater than the age- and sex-matched reference concentration; and
(iv) generate a network control signal to trigger an interruptive clinical alert on a clinical endpoint device based on the detected skeletal growth failure state.

14. The CDS system of claim 13, wherein the clinical logic gate detects a skeletal growth failure state when the plasma NT-proCNP concentration is equal to or greater than 2.0 standard deviations above the age- and sex-matched reference concentration.

15. The CDS system of claim 13, wherein the network communication interface is configured to asynchronously ingest the laboratory data packets into the clinical data repository.

16. The CDS system of claim 15, wherein the network control signal modifies a physical data structure within the clinical data repository by automatically appending a classification tag to an electronic medical record of the patient, and transmitting an asynchronous network control signal to a clinical endpoint device to render the interruptive clinical alert.

17. The CDS system of claim 13, wherein the network communication interface is configured to access the clinical data repository.

18. The CDS system of claim 17, wherein the network control signal is configured to modify rendering instructions of a graphical user interface (GUI) to overlay the interruptive clinical alert on an electronic medical record.

19. The CDS system of claim 18, wherein the instructions further cause the system to transmit an application programming interface (API) request to persistently store a classification tag in the clinical data repository.

20. (canceled)

21. The CDS system of claim 13, wherein the age- and sex-matched reference concentration comprises the mean concentration of NT-proCNP polypeptide in age- and sex-matched healthy individuals.
